SEQUENCE LISTING (64 sequences)

SEQ ID NO: 1 - DNA, 1739 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatag

SEQ ID NO: 2 - protein, 561 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 1344 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatag

SEQ ID NO: 4 - protein, 202 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGNISWTIPAISLSQIHSLVR

SEQ ID NO: 5 - DNA, 1344 bp, Homo sapiens; identical to SEQ ID NO: 3

SEQ ID NO: 6 - protein, 202 aa, Homo sapiens; identical to SEQ ID NO: 4

SEQ ID NO: 7 - DNA, 736 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcgttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatag

SEQ ID NO: 8 - protein, 164 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFVLVAVTVIVGHARVQV

SEQ ID NO: 9 - DNA, 2297 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatag

SEQ ID NO: 10 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 11 - DNA, 903 bp, artificial
  SIRT 1 2/9 optimized for recombinant expression
actcatctcagaagaggatctgaatcataccggtcatcatcaccatcaccatgagctcgccgatgaggcggcattggccctacagcctggcggatctcccagtgcagccggagcagatcgcgaggccgcttcctcacccgcaggggaaccccttcggaagagaccgcgcagagatgggcctggtctcgaacggagcccaggagaacctggcggtgcagctccagagcgtgaagtacccgctgccgcaagagggtgccctggtgccgcagctgccgcgctctggcgagaggccgaagccgaggccgctgcagccggaggcgaacaggaggctcaagccacagctgcagccggggaaggcgacaatggacccgggttacagggcccaagcagggaaccaccgcttgcggacaacctgtatgacgaggacgatgacgatgagggcgaggaagaggaggaggctgctgccgccgcgattggctacagggacaacctgctgttcgtgctggtggctgtgaccgtgatcgtcggtcacgccagggttcaggtctgaaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagaagctttcgtcgacgatatccagcacagtggcggccgctcgagtctagagggcccgcggttcgaacaaaaactcatctcagaagaggatctgaatatgcataccggtcatcatcaccatcaccattgagtttaaacccgctgatcagcctcgactgtgccttcta

SEQ ID NO: 12 - RNA, 19 nt, Artificial
  SIRT 1 siRNA specific for full length SIRT 1
acuuugcuguaacccugua

SEQ ID NO: 13 - RNA, 19 nt, Artificial
  siRNA specific for SIRT1-8
uaauuccaaguaaucagua

SEQ ID NO: 14 - RNA, 19 nt, Artificial
  siRNA specific for SIRT1-3/4
cacggauaggaaauauauc

SEQ ID NO: 15 - RNA, 19 nt, Artificial
  siRNA specific for SIRT1-9
ccuucuguucguucuugug

SEQ ID NO: 16 - DNA, 20 bp, artificial
  SIRT 1 forward primer
gggatggtatttatgctcgc

SEQ ID NO: 17 - DNA, 23 bp, Artificial
  SIRT 1 reverse primer
aagaggtgtgggtggcaactctg

SEQ ID NO: 18 - DNA, 20 bp, Artificial
  SIRT1-8 forward primer
gggatggtatttatgctcgc

SEQ ID NO: 19 - DNA, 21 bp, Artificial
  SIRT1-8 reverse primer
aacagatactgattacttgga

SEQ ID NO: 20 - DNA, 21 bp, Artificial
  SIRT1-3/4 forward primer
ccaaggccacggataggaaat

SEQ ID NO: 21 - DNA, 23 bp, Artificial
  SIRT 1-3/4 reverse primer
aagaggtgtgggtggcaactctg

SEQ ID NO: 22 - DNA, 21 bp, Artificial
  SIRT1-3/4/8 forward primer
ccaaggccacggataggaaat

SEQ ID NO: 23 - DNA, 21 bp, Artificial
  SIRT1-3/4/8 reverse primer
aacagatactgattacttgga

SEQ ID NO: 24 - DNA, 19 bp, Artificial
  SIRT1-2/9 forward primer
ataaccttctgttcgttct

SEQ ID NO: 25 - DNA, 25 bp, Artificial
  SIRT1-2/9 reverse primer
ctatgatttgtttgatggatagttc

SEQ ID NO: 26 - DNA, 21 bp, Artificial
  SIRT 1 full length and SIRT 3/4 forward primer
ctaattccaagttccataccc

SEQ ID NO: 27 - DNA, 21 bp, Artificial
  SIRT1 full length and SIRT1 3/4 reverse primer
ctgaagaatctggtggtgaag

SEQ ID NO: 28 - DNA, 21 bp, Artificial
  SIRT1-8 and SIRT1-3/4/8 forward primer
ccaaggccacggataggaaat

SEQ ID NO: 29 - DNA, 22 bp, Artificial
  SIRT1-8 and SIRT1-3/4/8 reverse primer
ctatgttctgggtatagttgcg

SEQ ID NO: 30 - DNA, 21 bp, Artificial
  PAX 6 5a forward primer
gcagatgcaaaagtccaagtg

SEQ ID NO: 31 - DNA, 20 bp, Artificial
  PAX6 5a reverse primer
cagccatcttgcgtaggttg

SEQ ID NO: 32 - DNA, 19 bp, Artificial
  PAX6 -5a forward primer
cccgaattctgcaggtgtc

SEQ ID NO: 33 - DNA, 20 bp, Artificial
  PAX6 -5a reverse primer
cagccatcttgcgtaggttg

SEQ ID NO: 34 - RNA, 19 nt, Artificial
  siRNA specific for PAX 6
caucaguuccaacggagaa

SEQ ID NO: 35 - RNA, 19 nt, Artificial
  PAX6 5a specific siRNA
agaugcaaaaguccaagug

SEQ ID NO: 36 - RNA, 19 nt, Artificial
  PAX6 -5a specific siRNA
auucugcagguguccaacg

SEQ ID NO: 37 - DNA, 19 bp, Artificial
  1 forward primer
ataaccttctgttcgttct

SEQ ID NO: 38 - DNA, 23 bp, Artificial
  8 reverse primer
aagaggtgtgggtggcaactctg

SEQ ID NO: 39 - DNA, 25 bp, Artificial
  10 reverse primer
ctatgatttgtttgatggatagttc

SEQ ID NO: 40 - DNA, 18 bp, Artificial
  APAF1 forward primer
tgcgctgctctgccttct

SEQ ID NO: 41 - DNA, 21 bp, Artificial
  APAF1 reverse primer
ccatgggtagcagctccttct

SEQ ID NO: 42 - DNA, 18 bp, Artificial
  BGH primer
tagaaggcacagtcgagg

SEQ ID NO: 43 - DNA, 20 bp, Artificial
  CUGBP1 forward primer
gataggagccaaaacccgcc

SEQ ID NO: 44 - DNA, 20 bp, Artificial
  CUGBP1 reverse primer
tcacaaatgcacaacctcgg

SEQ ID NO: 45 - DNA, 20 bp, Artificial
  CUGBP2 forward primer
gtgaaaagtccaacgctgtg

SEQ ID NO: 46 - DNA, 19 bp, Artificial
  CUGBP2 reverse primer
ccaggtggcagtgttgagc

SEQ ID NO: 47 - DNA, 21 bp, Artificial
  GAPDH forward primer
cggagtcaacggatttggtcg

SEQ ID NO: 48 - DNA, 21 bp, Artificial
  GAPDHG reverse primer
agccttctccatggtggtgaa

SEQ ID NO: 49 - DNA, 20 bp, Artificial
  HO1 forward primer
ccagtgccaccaagttcaag

SEQ ID NO: 50 - DNA, 20 bp, Artificial
  HO1 reverse primer
cagctcctgcaactcctcaa

SEQ ID NO: 51 - DNA, 21 bp, Artificial
  IGFBP3 forward primer
gacagaatatggtccctgccg

SEQ ID NO: 52 - DNA, 19 bp, Artificial
  IGFBP3 reverse primer
ttggaagggcgacactgct

SEQ ID NO: 53 - DNA, 26 bp, Artificial
  MMP1 forward primer
tttgatgtaccctagctacaccttca

SEQ ID NO: 54 - DNA, 27 bp, Artificial
  MMP1 reverse primer
aaaggttagcttactgtcacatgcttt

SEQ ID NO: 55 - DNA, 21 bp, Artificial
  P53 specific primer
atggaggagccgcagtcagat

SEQ ID NO: 56 - DNA, 21 bp, Artificial
  p53 specific primer
tcagtctgagtcaggcccttc

SEQ ID NO: 57 - DNA, 22 bp, Artificial
  SC35 forward primer
acaacctgacctaccgcacctc

SEQ ID NO: 58 - DNA, 20 bp, Artificial
  SC35 reverse primer
gacaccgctccttccttcag

SEQ ID NO: 59 - DNA, 20 bp, Artificial
  SEx specific primer
gggatggtatttatgctcgc

SEQ ID NO: 60 - DNA, 33 bp, Artificial
  SIRT 1 primer
gcgatatctgatttgtttgatggatagttcatg

SEQ ID NO: 61 - DNA, 23 bp, Artificial
  TBP forward primer
caggagccaagagtgaaagaaca

SEQ ID NO: 62 - DNA, 21 bp, Artificial
  TBP reverse primer
agctggaaaacccaattctgt

SEQ ID NO: 63 - DNA, 19 bp, Artificial
  tPA forward primer
atgcccgattcagaagagg

SEQ ID NO: 64 - DNA, 20 bp, Artificial
  tPA reverse primer
gacaggcactgagtggcact

(19) United States
(12) Patent Application Publication
(10) Pub. No.: US 2012/0178796 A1
(43) Pub. Date: Jul. 12, 2012

(54) SPLICE VARIANTS

(76) Inventors: Josephine Milner, York (GB); Zahid Hussain Shah, York (GB)

(21) Appl. No.: 13/497,038

(22) PCT Filed: Sep. 22, 2010
(86) PCT No.: PCT/GB10/01786

§ 371 Date: Mar. 19, 2012

(30) Foreign Application Priority Data
Sep. 28, 2009 (GB) 0916889.9

(51) Int. Cl. A61K 31/713; C12N 5/10; C12N 1/21; C07H 21/02; C07K 14/47; C12N 15/12; C07H 21/04; C12Q 1/68; C12N 15/63

(52) U.S. Cl. 514/44 A; 435/6.12; 435/325; 435/377; 435/252.3; 435/320.1; 530/350; 536/23.5; 536/24.33; 536/24.5

(57) ABSTRACT

We disclose the isolation and characterization of sirtuin 1 [SIRT1] splice variants.

The invention relates to the isolation and characterization of sirtuin 1 [SIRT1] splice variants.

The ability of mammalian cells to withstand metabolic and genotoxic stress involves SIRT1, a class III histone de-acetylase able to regulate gene expression at several levels. Thus, deacetylation of linker histone H1 by SIRT1 enables heterochromatin formation and associated gene silencing. SIRT1 also deacetylates core histone H3 and recruitment of SIRT1 to specific promoters results in selective gene silencing. In addition SIRT1 targets several non-histone transcription regulators including the tumour suppressor p53. De-acetylation of p53 by SIRT1 down-regulates the pro-apoptotic p53 stress response. SIRT1 and p53 thus counterbalance the cellular response to stress. This balance is dependent upon cellular levels of SIRT1 and p53 since over-expression favours cell survival or apoptosis respectively. Expression levels of p53 and SIRT1 therefore require stringent control. For p53 this is largely achieved through regulation of p53 protein stability. For SIRT1 a transcriptional feed-back mechanism operates in which SIRT1 forms a complex with the transcription repressor hypermethylated in cancer 1 (HIC1) and selectively suppresses transcription from the SIRT1 promoter. SIRT1 expression is also regulated at the level of mRNA stability via the RNA-binding protein HuR which binds and stabilises SIRT1 mRNA. The human SIRT 1 gene has a complex intron/exon structure comprising 9 exons.

This disclosure relates to the identification and characterization of novel human SIRT 1 splice variants with altered biological properties. We disclose that SIRT1 generates variant RNAs: SIRT1-Full Length [FL], SIRT1-delta exon 8 (SIRT1-Δ8); SIRT1-Δ3/4; SIRT1-Δ3/4/8 and SIRT1-Δ2-9. SIRT1-Δ2-9 RNA appears to predominate in cancer tissues. SIRT1-Δ8 is basally expressed throughout normal and cancerous tissues and exogenous SIRT1-Δ8 interacts with p53 and AROS. SIRT1-FL, but not SIRT1-Δ8, is required for cancer-specific survival. In undifferentiated cells SIRT1-FL and SIRT1-Δ8 appear to exert opposing effects upon PAX-6, a stem cell factor which directs neuronal differentiation and gluconeogenesis. Alternative splicing is thus a crucial consideration for SIRT1-based strategies to treat diabetes, neuropathies and cancer.

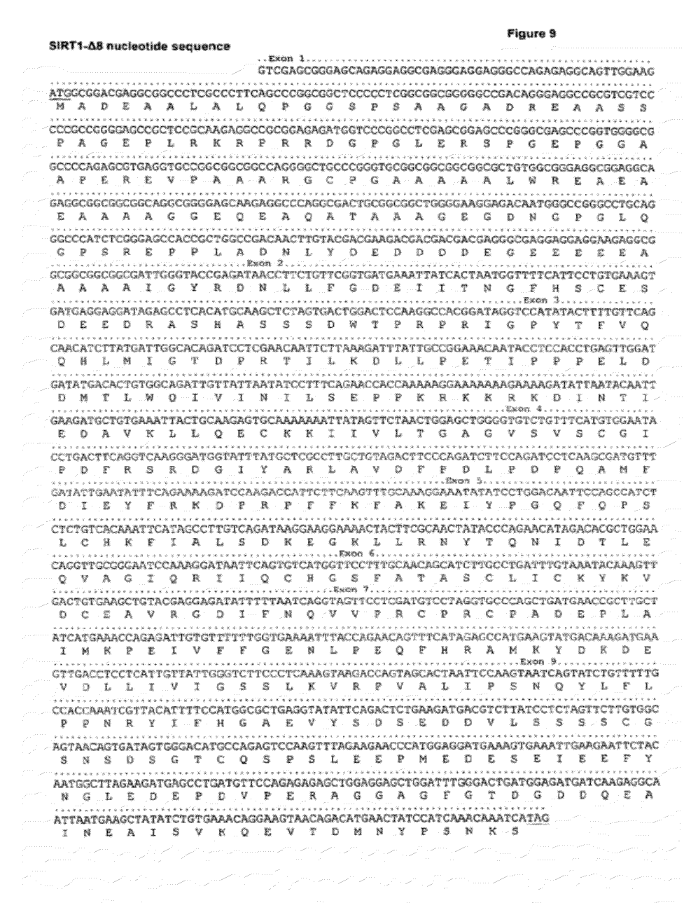

According to an aspect of the invention there is provided an isolated nucleic acid molecule comprising or consisting of a nucleotide sequence selected from the group consisting of:
i) a nucleotide sequence as represented in FIG. 9, 10, 11 or 12;
ii) a nucleotide sequence wherein said sequence is degenerate as a result of the genetic code to the nucleotide sequence defined in (i);
iii) a nucleotide sequence that encodes an amino acid sequence as represented in FIG. 9, 10, 11 or 12 wherein said amino acid sequence is modified by addition deletion or substitution of at least one amino acid residue.

According to an aspect of the invention there is provided an isolated polypeptide comprising an amino acid sequence as represented 9, 10, 11 or 12, or a variant polypeptide wherein said variant polypeptide is modified by addition deletion or substitution of at least one amino acid residue and wherein said variant polypeptide has modified activity

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 85% identity, more preferably at least 90% identity, even more preferably at least 95% identity, still more preferably at least 97% identity, and most preferably at least 99% identity with the full length amino acid sequences illustrated herein.

According to an aspect of the invention there is provided a vector that includes a nucleic acid molecule according to the invention.

Preferably said vector is an expression vector adapted for prokaryote or eukaryote expression.

According to a further aspect of the invention there is provided a cell transformed or transfected with a nucleic acid molecule or vector according to the invention.

In a preferred embodiment of the invention said cell is a eukaryote cell; preferably a mammalian cell.

In an alternative preferred embodiment of the invention said cell is a prokaryote cell; preferably a microbial cell, e.g. a bacterial cell.

In a preferred embodiment of the invention said cell is stably transfected.

In an alternative preferred embodiment of the invention said cell is transiently transfected.

According to a further aspect of the invention there is provided a small interfering RNA [siRNA] molecule wherein said siRNA molecule is specific for at least one SIRT 1 spliced variant as represented by the nucleotide sequences in FIG. 9, 10, 11 or 12.

In a preferred embodiment of the invention said siRNAs molecule specifically bind the spliced junctions of said spliced variant.

A technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated. The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

In our co-pending application U.S. Ser. No. 11/915,147 we disclose a modified siRNA-DNA construct (termed ‘crook’ siRNA). The transfection into mammalian cells of crook siRNA induces selective mRNA knock-down equivalent to its unmodified siRNA counterpart. This bi-functional siRNA is described in U.S. Ser. No. 11/915,147, which is incorporated by reference in its entirety.

In a preferred embodiment of the invention there is provided a siRNA molecule comprising a first part that comprises a duplex ribonucleic acid (RNA)molecule and a second part that comprises a single stranded deoxyribonucleic acid (DNA) molecule wherein said single stranded DNA molecule comprises a 3′ terminal nucleic acid sequence wherein said sequence is adapted over at least part of its length to anneal by complementary base pairing to a part of said single stranded DNA to form a double stranded DNA structure.

Typically the single stranded DNA molecule is at least 7 nucleotides in length. Preferably said single stranded DNA molecule is between 10-40 nucleotide bases in length, more preferably 15-30 nucleotides in length.

In a preferred embodiment of the invention said duplex RNA molecule is at least 18 base pairs in length.

In a further preferred embodiment of the invention said duplex RNA molecule is between 19bp and 1000bp in length. More preferably the length of said duplex RNA molecule is at least 30bp; at least 40bp; at least 50bp; at least 60bp; at least 70bp; at least 80bp; or at least 90bp.

In a yet further preferred embodiment of the invention said duplex RNA molecule is at least 100bp; at least 200bp; at least 300bp; at least 400bp; at least 500bp; at least at least 600bp; at least 700bp; at least 800bp; at least 900bp; or at least 1000bp in length.

Preferably said duplex RNA molecule is between 18bp and 29bp in length. More preferably still said duplex RNA molecule is between 21 by and 27bp in length. Preferably said duplex RNA molecule is about 21 by in length.

In a preferred embodiment of the invention said siRNA molecule is selected from the group consisting of:

i)
ACUUUGCUGUAACCCUGUA;
ii)
UAAUUCCAAGUAAUCAGUA;
iii)
CACGGAUAGGAAAUAUAUC;
and
iv)
CCUUCUGUUCGUUCUUGUG.

In a preferred embodiment of the invention said siRNA includes modified nucleotides.

The term “modified” as used herein describes a nucleic acid molecule in which;
i) at least two of its nucleotides are covalently linked via a synthetic internucleoside linkage (i.e., a linkage other than a phosphodiester linkage between the 5′ end of one nucleotide and the 3′ end of another nucleotide). Alternatively or preferably said linkage may be the 5′ end of one nucleotide linked to the 5′ end of another nucleotide or the 3′ end of one nucleotide with the 3′ end of another nucleotide; and/or
ii) a chemical group, such as cholesterol, not normally associated with nucleic acids has been covalently attached to the double stranded nucleic acid.
iii) Preferred synthetic internucleoside linkages are phosphorothioates, alkylphosphonates, phosphorodithioates, phosphate esters, alkylphosphonothioates, phosphoramidates, carbamates, phosphate triesters, acetamidates, peptides, and carboxymethyl esters.

The term “modified” also encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′- fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5 carboxymethylaminomethyl uracil; dihydrouracil; inosine; N6-isopentyl-adenine; I-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine;

5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Modified double stranded nucleic acids also can include base analogs such as C-5 propyne modified bases (see Wagner et al., Nature Biotechnology 14:840-844, 1996).

According to a further aspect of the invention there is provided a pharmaceutical composition comprising a siRNA molecule according to the invention and including an excipient or carrier.

When administered the compositions of the present invention are administered in pharmaceutically acceptable preparations. Such preparations may routinely contain pharmaceutically acceptable concentrations of salt, buffering agents, preservatives, compatible carriers and supplementary anti-cancer agents.

The compositions of the invention can be administered by any conventional route, including injection or by gradual infusion over time. Treatment may be topical or systemic. The administration may, for example, be oral, intravenous, intraperitoneal, intramuscular, intracavity, subcutaneous, transdermal, transepithelial or intra bone marrow administration.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an agent according to the invention for producing the desired response in a unit of weight or volume suitable for administration to a patient.

The doses of the siRNA according to the invention administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of siRNA of between 1nM-1μM generally will be formulated and administered according to standard procedures. Preferably doses can range from 1 nM-500 nM, 5 nM-200 nM, and 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents' (e.g. anti-inflammatory agents such as steroids, non-steroidal anti-inflammatory agents). When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” in this context denotes an organic or inorganic ingredient, natural or synthetic, with which the active ingredient is combined to facilitate the application, (e.g. liposome or immuno-liposome). The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion or as a gel. Compositions may be administered as aerosols and inhaled.

Compositions suitable for parenteral administration conveniently comprise a sterile aqueous or non-aqueous preparation of agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to an aspect of the invention there is provided a method to diagnose cancer in a subject comprising:
i) providing an isolated biological sample to be tested;
ii) forming a preparation comprising said sample and one or more oligonucleotide primer pairs adapted to anneal to a nucleic acid molecule comprising a nucleic acid sequence as represented in FIG. 9, 10, 11 or 12; a thermostable DNA polymerase, deoxynucleotide triphosphates and co-factors;
iii) providing polymerase chain reaction conditions sufficient to amplify said nucleic acid molecule[s];
iv) analysing the amplified product[s] of said polymerase chain reaction for the presence or absence of amplified product[s]; and optionally
v) comparing the amplified product[s] with a normal matched control.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Further examples include lung cancer for example small cell lung carcinoma or a non-small cell lung cancer. Other classes of lung cancer include neuroendocrine cancer, sarcoma and metastatic cancers of different tissue origin.

In a preferred method of the invention the oligonucleotide primer pairs include oligonucleotide primers selected from the group consisting of the nucleotide sequences:

i)
GGGATGGTATTTATGCTCGC
and
AAGAGGTGTGGGTGGCAACTCTG;
ii)
GGGATGGTATTTATGCTCGC
and
AACAGATACTGATTACTTGGA;
iii)
CCAAGGCCACGGATAGGAAAT
and
AAGAGGTGTGGGTGGCAACTCTG;
iv)
CCAAGGCCACGGATAGGAAAT
and
AACAGATACTGATTACTTGGA;
v)
ATAACCTTCTGTTCGTTCT
and
CTATGATTTGTTTGATGGATAGTTC;
vi)
CTAATTCCAAGTTCCATACCC
and
CTGAAGAATCTGGTGGTGAAG;
and
vii)
CCAAGGCCACGGATAGGAAAT
and
CTATGTTCTGGGTATAGTTGCG.

In a preferred method of the invention said comparison includes a quantitative and/or qualitative analysis of the expression of two or more SIRT 1 spliced variant relative to a normal matched control.

According to a further aspect of the invention there is provided a kit comprising at least one variant specific primer pair selected from the group consisting of:

i)
GGGATGGTATTTATGCTCGC
and
AAGAGGTGTGGGTGGCAACTCTG;
ii)
GGGATGGTATTTATGCTCGC
and
AACAGATACTGATTACTTGGA;
iii)
CCAAGGCCACGGATAGGAAAT
and
AAGAGGTGTGGGTGGCAACTCTG;
iv)
CCAAGGCCACGGATAGGAAAT
and
AACAGATACTGATTACTTGGA;
v)
ATAACCTTCTGTTCGTTCT
and
CTATGATTTGTTTGATGGATAGTTC;
vi)
CTAATTCCAAGTTCCATACCC
and
CTGAAGAATCTGGTGGTGAAG;
and
vii)
CCAAGGCCACGGATAGGAAAT
and
CTATGTTCTGGGTATAGTTGCG.

In a preferred embodiment of the invention said kit further includes reagents required for polymerase chain reaction amplification of SIRT 1 spliced variant RNA.

According to a further aspect of the invention there is provided a progentitor retinal pigmented epithelial [PRPE] cell which cell is modified wherein said modified cell has reduced or undetectable levels of SIRT 1.

In a preferred embodiment of the invention said PRPE cell is modified by transfection a siRNA that reduces expression of SIRT 1.

In a preferred embodiment of the invention said cell is stably transfected.

In an alternative preferred embodiment of the invention said cell is transiently transfected.

In a further preferred embodiment of the invention said siRNA is expressed by said PRPE cell.

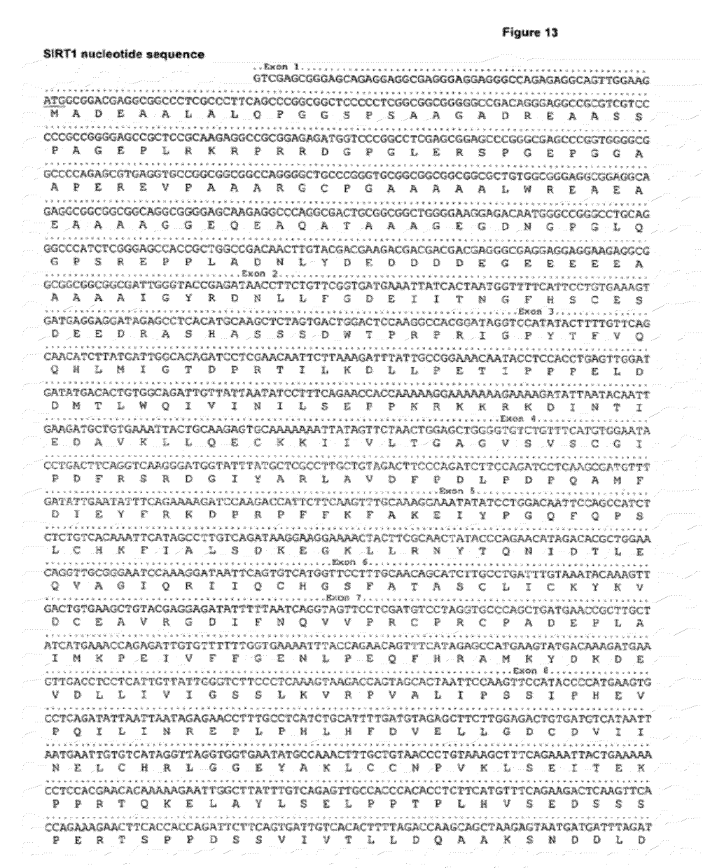

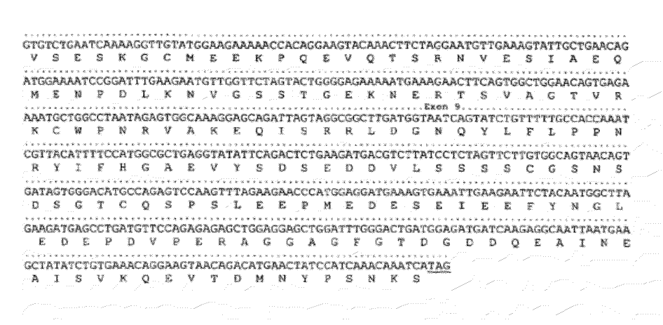

In a preferred embodiment of the invention SIRT 1 is encoded by a nucleic acid molecule comprising a nucleotide sequence as represented in FIG. 13.

In a preferred embodiment of the invention said siRNA is designed with reference to the nucleotide sequence as represented in FIG. 13.

According to a further aspect of the invention there is provided a method to enhance the differentiation of a progenitor retinal pigmented epithelial cell comprising:
i) providing a cell culture preparation comprising: a PRPE cell according to the invention and a cell culture medium; and
i) providing cell culture conditions that enhance the differentiation of PRPE cells.

According to a further aspect of the invention there is provided an agent that inhibits the expression or activity of SIRT 1 for use in the differentiation of PRPE cells.

In a preferred embodiment of the invention said agent is a siRNA.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

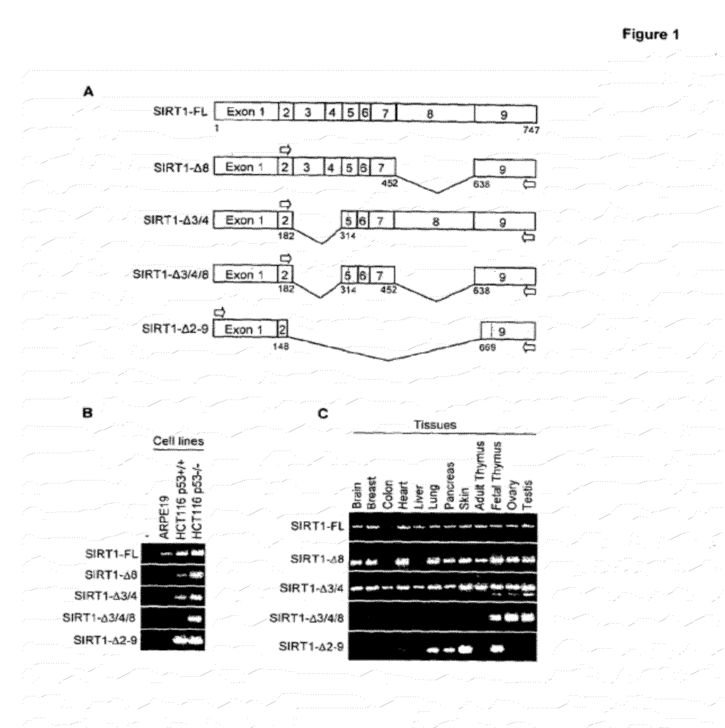

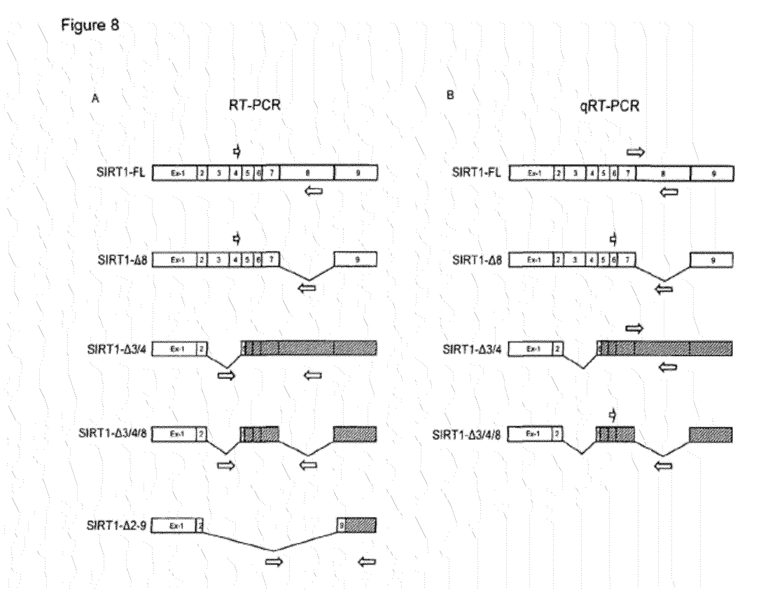

FIG. 1 Identification of novel human SIRT1 splice variants and their expression in human tissues. (A) Schematic showing the exon structure of the alternatively spliced human SIRT1 variants. Exons denoted 1 to 9. Arrows indicate primer pairs used which led to the discovery of the splice variants (see Supplementary FIG. 1). Shaded areas indicate non-coding regions. Numbers at splice junctions show corresponding amino acid positions in SIRT1-FL. (B,C) Expression levels of the individual SIRT1 splice variants in non-cancer ARPE19 cells and HCT116 colorectal carcinoma cells (B) and in a range of normal human tissues (C). Splice variant-specific primer pairs were used in RT-PCR on total RNA from indicated tissue samples. The location and sequence of primers used is given in FIG. 8 and Supplementary Table 2;

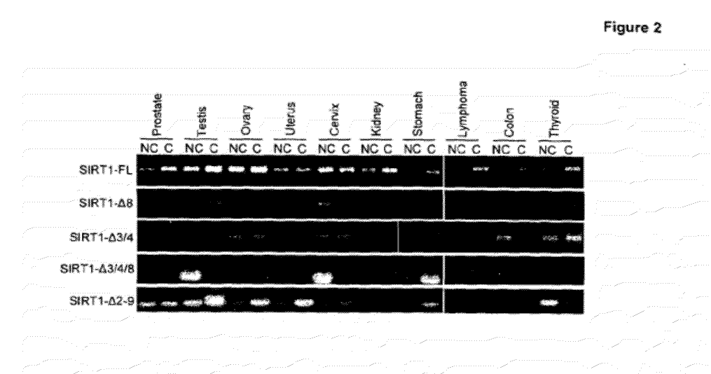

FIG. 2 Expression levels of SIRT1 splice variants in total RNA from paired cancer (C) and non-cancer (NC) human tissue samples;

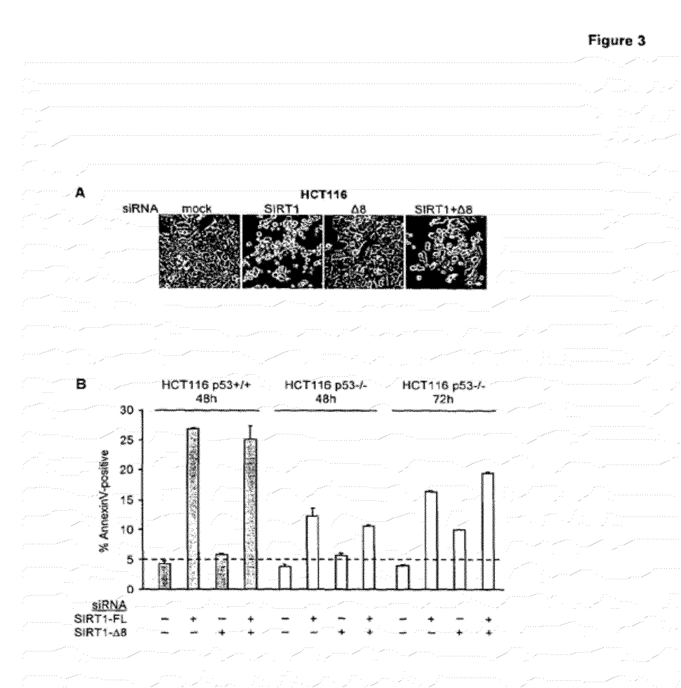

FIG. 3 Differential effects of FL-SIRT1 and SIRT1-Δ8 on cancer cell survival. (A) Micrographs showing the effects of FL-SIRT1 and SIRT1-Δ8 silencing on HCT116 p53+/+ colorectal carcinoma cells. (B) Quantitation of apoptosis by annexin V staining following silencing with the indicated siRNAs;

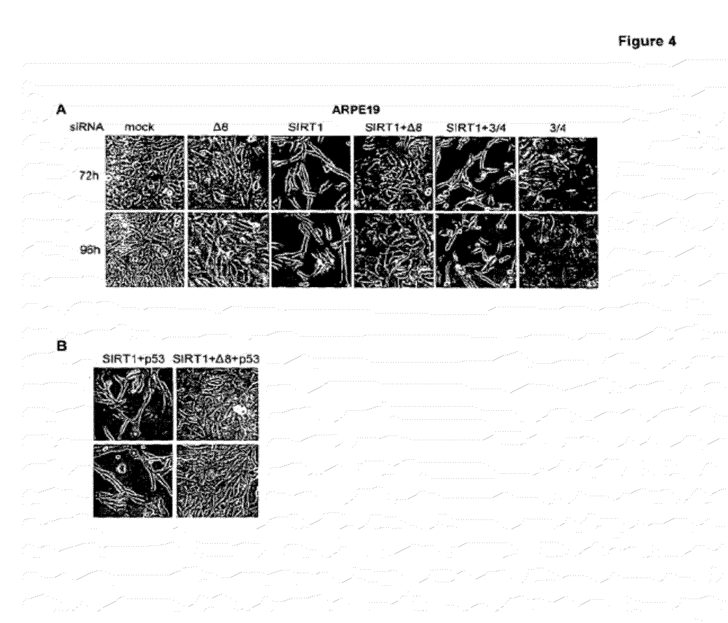

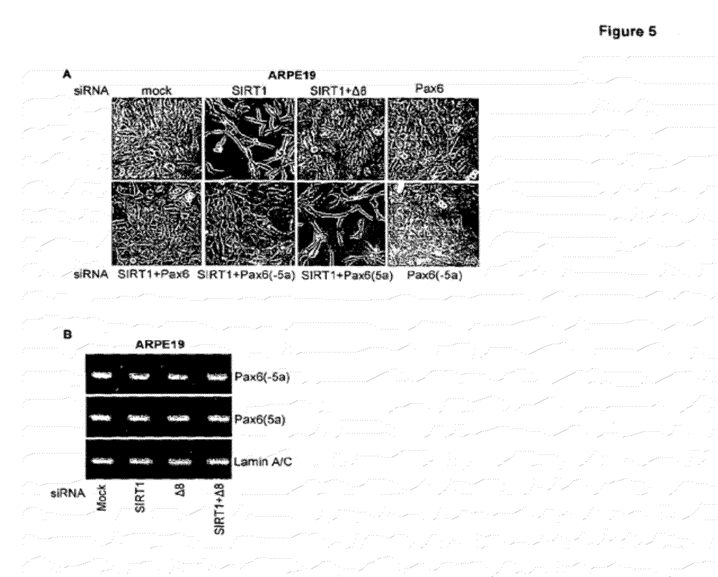

FIG. 4 Effects of RNAi silencing of SIRT1 splice variants on neuronal differentiation of human ARPE19 retinal epithelial cells. (A) Micrographs showing a morphological phenotype resembling neuronal differentiation that is selectively induced by FL-SIRT1 silencing and selectively rescued by SIRT1-Δ8 co-silencing. (B) Effect of p53 status on neuronal differentiation induced by FL SIRT1 silencing;

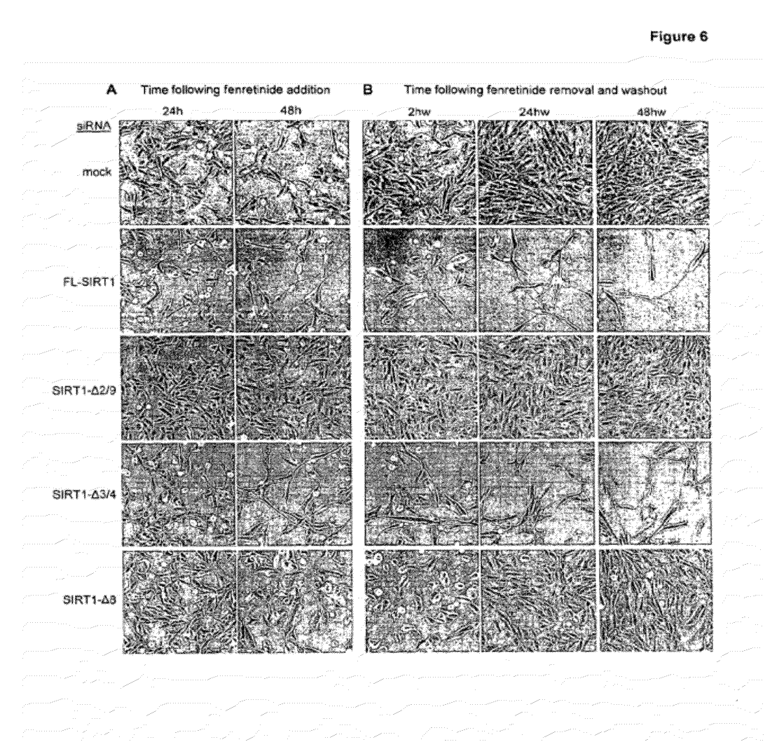

FIG. 5 Pax6 is required for neuronal differentiation constitutively inhibited by FL-SIRT1 and is positively regulated by SIRT1-Δ8. (A) Micrographs showing the effect of co-silencing Pax6 with FL-SIRT1 and individually co-silencing the Pax6 splice variants Pax6(-5a) and Pax6(5a) with FL-SIRT1. (B) RT-PCR analysis showing the effects of FL-SIRT1 and SIRT1-Δ8 silencing on Pax6(-5a) and Pax6(5a) mRNA expression;

FIG. 6 Differential requirements of the SIRT1 splice variants for fenretinide-induced neuronal differentiation. (A) Cell micrographs showing the effects of SIRT1 splice variant silencing on fenretidine-induced neuronal differentiation 24 and 48 h following fenretidine addition. (B) Effects of silencing on maintenance of the neuronal differentiation phenotype following fenretinide removal and washout (see methods);

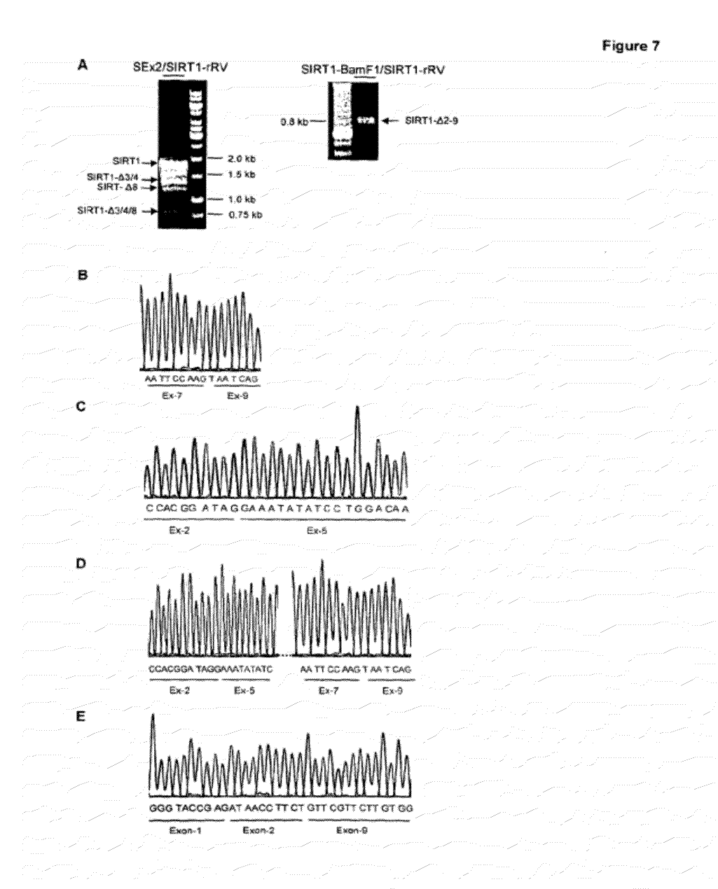

FIG. 7 Identification and sequencing of human SIRT1 splice variants. (A) Total RNA from HCT116 p53-/- cells was reverse transcribed and PCR amplified using the indicated primers (see methods) and analysed by agarose gel electrophoresis. Arrows indicate amplified DNA fragments corresponding to SIRT1 splice variants. (B-E) Sequencing of SIRT1-L8 (B), SIRT1-Δ3/4 (C); SIRT1-Δ3/4/8 (D) and SIRT1-Δ2-9 (E) across the indicated novel splice junctions;

FIG. 8 (A) Schematic showing location of primers for detection of individual SIRT1 splice variants by RT-PCR. (B) Primer location for quantitative RT-PCR.

FIG. 9 Nucleotide and amino acid sequence of SIRT1-Δ8. Exons corresponding to FL-SIRT1 are indicated above the nucleotide sequence. Translation initation and stop codons are underlined. Amino acid sequence is indicated by the single letter code;

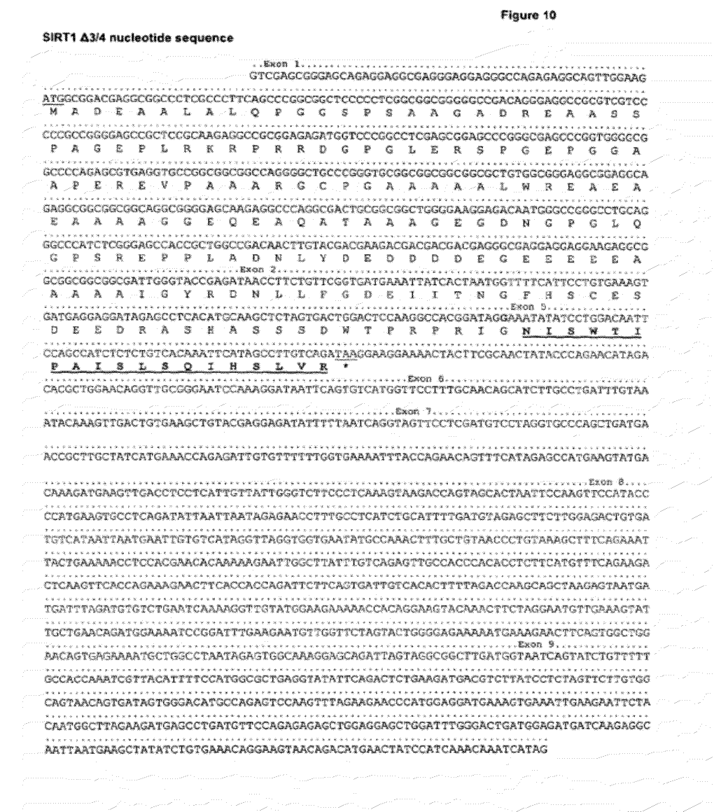

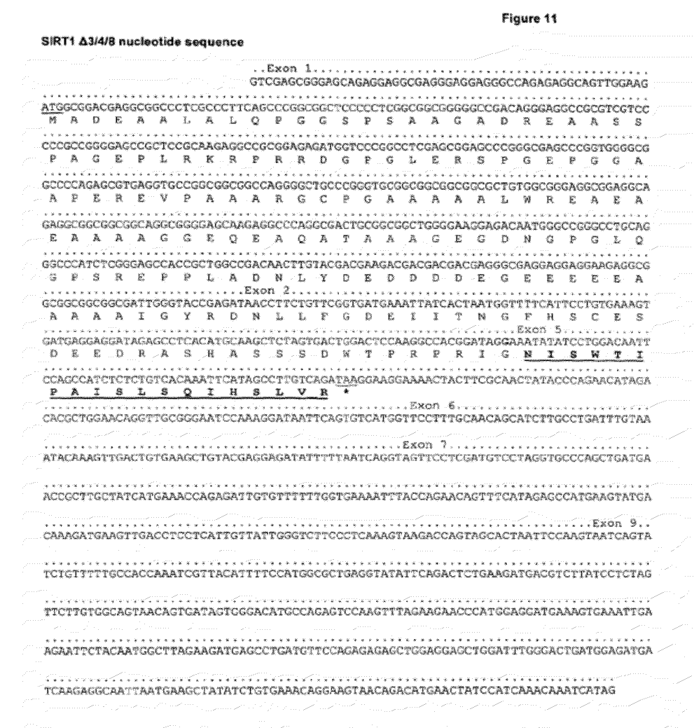

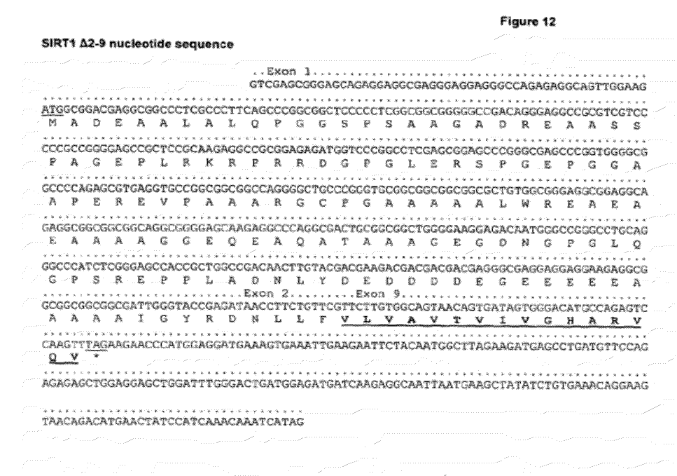

FIG. 10 Nucleotide and amino acid sequence of SIRT1-Δ3/4. A frameshift caused by splicing of exons 2 and 5 is indicated by the nucleotide highlighted in bold type. The resulting novel amino acid sequence is highlighted in bold type and underlined. The premature stop codon in exon 5 caused by the change in the reading frame is marked by an asterisk;

FIG. 11 Nucleotide and amino acid sequence of SIRT1-Δ3/4/8. A frameshift caused by splicing of exons 2 and 5 is indicated by the nucleotide highlighted in bold type. The resulting novel amino acid sequence is highlighted in bold type and underlined. The premature stop codon in exon 5 caused by the change in the reading frame is marked by an asterisk;

FIG. 12 Nucleotide and amino acid sequence of SIRT1-Δ2-9. A frameshift at the splice junction within exon 2 and exon 9 is indicated by the nucleotide highlighted in bold type. The resulting novel amino acid sequence is highlighted in bold type and underlined. The premature stop codon in exon 9 caused by the change in the reading frame is marked by an asterisk; and

FIG. 13 Nucleotide and amino acid sequence of FL-SIRT1. Exons are indicated above the nucleotide sequence. Translation initation and stop codons are underlined. Amino acid sequence is indicated by the single letter code.

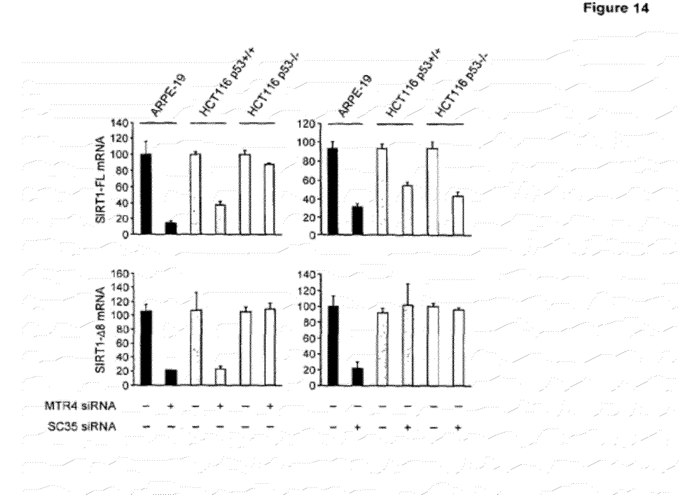

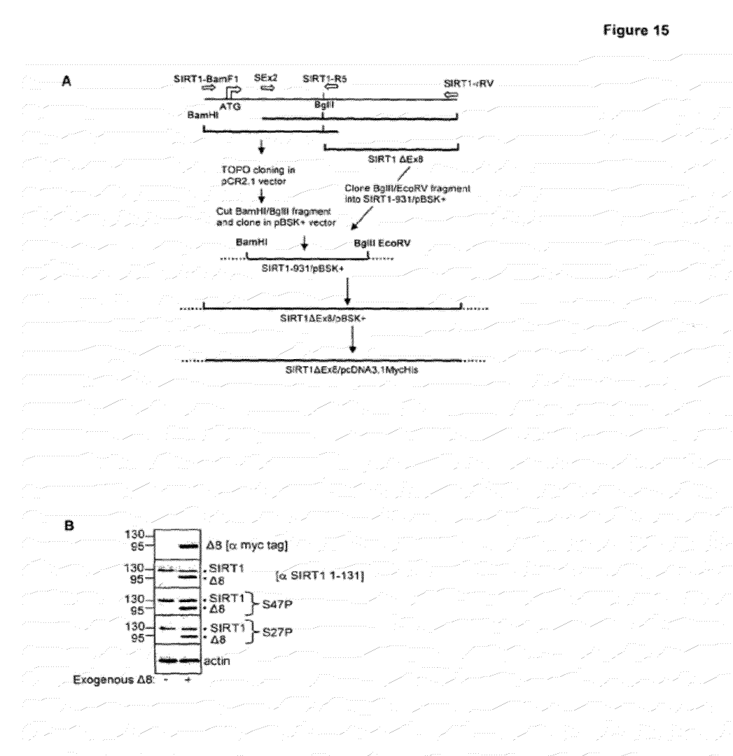

FIG. 14: Effects of silencing MTR4 and SC35 upon the expression levels of SIRT1 FL and SIRT1 D8 mRNAs as indicated. RNA interference was performed as detailed in the Materials and Methods. Cell lines=HCT116 p53+/+, HCT116 p53-/- and ARPE19 as indicated.

FIG. 15: Schematic outlining cloning strategy for the cloning of SIRT1 FL and of SIRT1 Δ 8 into a mammalian expression vector and showing primers and restriction sites. B. SIRT1-ΔExon8 is expressed as a protein and is also phosphorylated at S27 and S47. Myc-tagged SIRT1-ΔExon8 was transiently expressed in HCT116 cells and total cell lysates were probed with an array of antibodies. The Myc-epitope tag was probed with anti-c-Myc antibody (top panel; anti-myc 9E10, Santa Cruz) and detects only SIRT1-ΔExon8 (˜95 kDa). The lower panels were probed with SIRT1 antibodies and therefore detect endogenous SIRT1-FL (-116 kDa, upper band) and exogenous SIRT1-ΔExon8 (˜95 kDa, lower band): a C-terminal-specific SIRT1 antibody (residues 448-747, H300, Santa Cruz), a N-terminal-specific SIRT1 antibody (residues 1-131, Upstate), and phospho-specific antibodies for phosphorylated SIRT1-serine27 (S27) or phosphorylated SIRT1-serine47 (S47). Actin levels=loading equivalence.

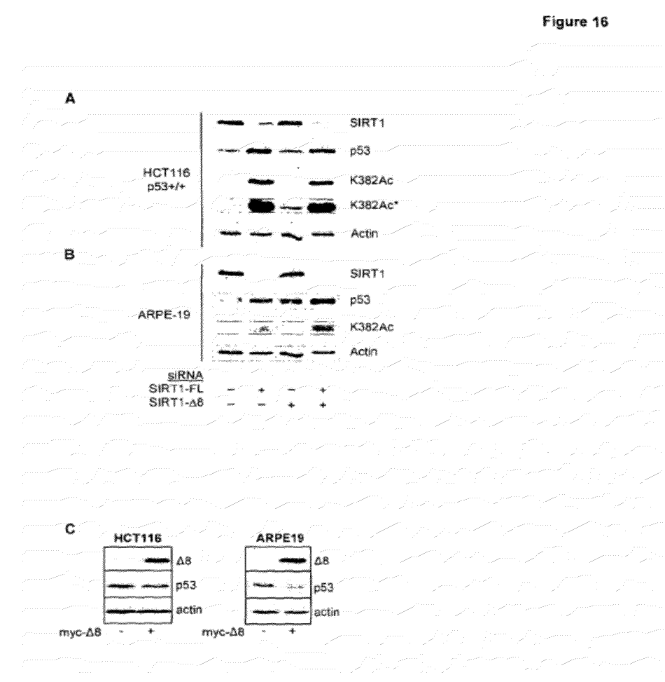

FIG. 16 Immunoblots showing effects of SIRT1 FL and SIRT1 D8 silencing upon p53 protein levels and acetylation status in (A) HCT116 and (B) ARPE19 cells as indicated. Actin=loading control. C=immunoblot showing effect of exogenous expression of SIRT1 D8 on endogenous p53 protein in HCT116 and ARPE19 cells as indicated.

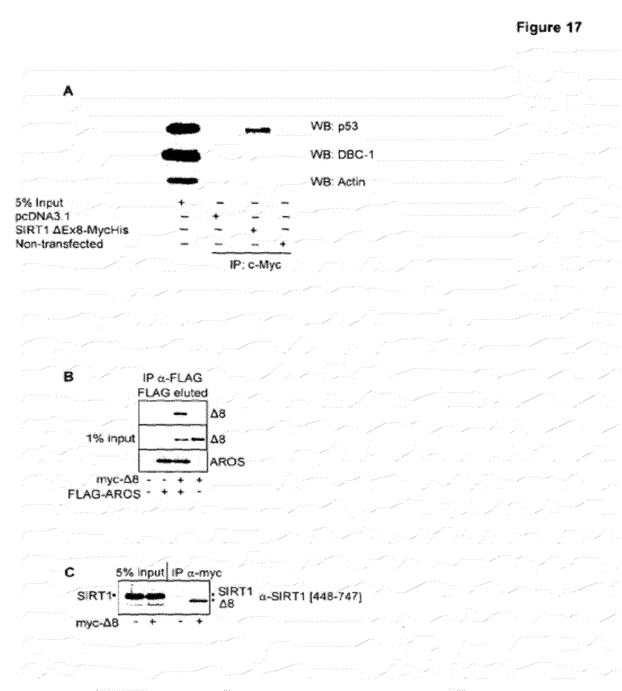

FIG. 17 A: SIRT1-ΔExon8 interacts with p53, but not DBC-1. SIRT1-ΔExon8 was exogenously expressed in HCT116 cells and immuno-precipitated. Eluates were probed for endogenous p53 (anti-p53 DO-1) or endogenous DBC-1 (anti-DBC-1) to assay for interactions with SIRT1-□Exon8 in vivo (Methods). B. SIRT1-ΔExon8 interacts with AROS. SIRT1-ΔExon8 and Flag-tagged AROS were exogenously expressed in HCT116 cells, and the Flag epitope was immuno-precipitated. In the eluate, AROS was detected by probing with an anti-AROS antibody (Methods), while the interaction with SIRT1-ΔExon8 was probed with anti-c-Myc antibody (9E10, Santa Cruz). C. SIRT1-ΔExon8 and SIRT FL proteins do not physically interact. SIRT1-ΔExon8 was exogenously expressed in HCT116 cells and cells were harvested 24 h post-transfection. Cell lysates were immuno-precipitated with anti-c-Myc antibody (9E10, Santa Cruz). Western-blotting was performed with a C-terminal anti-SIRT1 antibody (residues 448-747, H300, Santa Cruz) which detects both endogenous SIRT1-FL and SIRT1-ΔExon8 (see: FIG. 2a). * indicates a non-specific band;

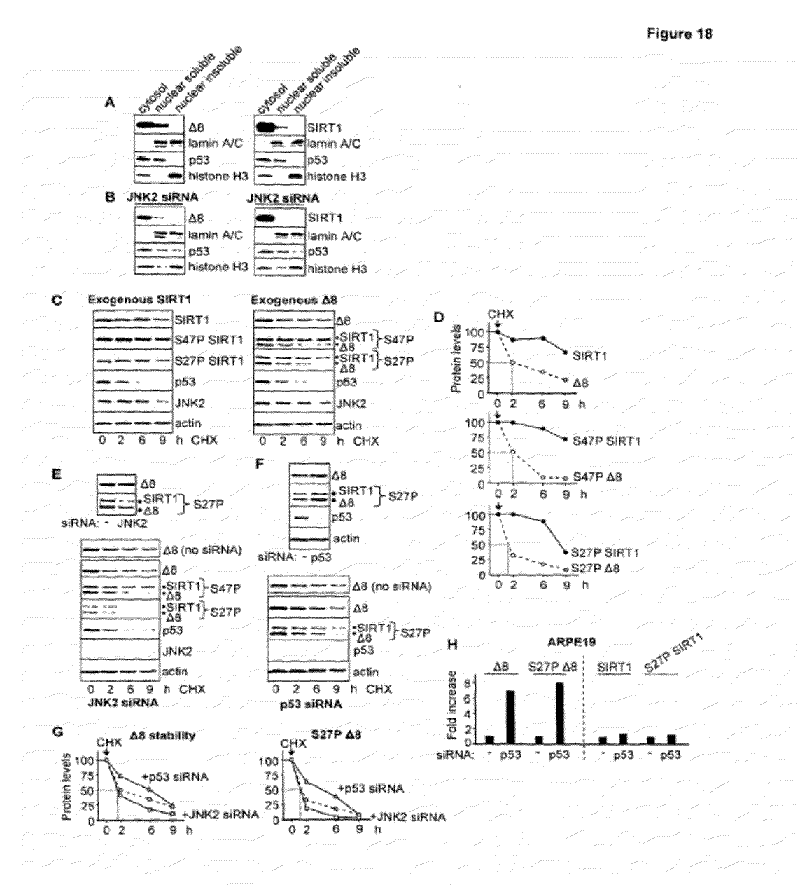

FIG. 18A: Cellular localisation of Δ8 and FL SIRT1 [in HCT116 p53++; by cell fractionation]. SIRT1 Δ8 and FL SIRT1 differ in their subcellular localisation [much higher proportion of cellular Δ8 localises to the nuclear soluble fraction compared with FL SIRT1 which is pedominantly cytoplasmic in HCT116 p53++ cells. Δ8 detectable in cytosol, nuclear soluble and nuclear insoluble fractions]. [Lamin A/C, histone H3 & p53 as fractionation controls]. Nuclear/chromatin functions for Δ8. B. JNK2 depletion alters the cellular localisation of Δ8 and FL SIRT1 with the translocation of the bulk of both Δ8 and FL SIRT1 to the cytosol. There is also a reduction in total levels of Δ8 and FL SIRT1.[Lamin A/C, histone H3 & p53 as fractionation controls. Alteration in p53 localisation—now detectable in nuclear insoluble fraction whereas under basal conditions was only detected in cytosol & nuclear soluble]. Suggests JNK2[kinase/signalling] can regulate the intracellular localisation of Δ8 and FL SIRT1, and that this may be involved in regulating Δ8 and FL SIRT1 protein turnover [JNK2 depletion may target Δ8 and FL SIRT1 for proteosomal-mediated degradation in the cytoplasm]. C—H Determination of protein half-life for Δ8 and FL SIRT1 following treatment with cyloheximide (CHX) and effects of p53;

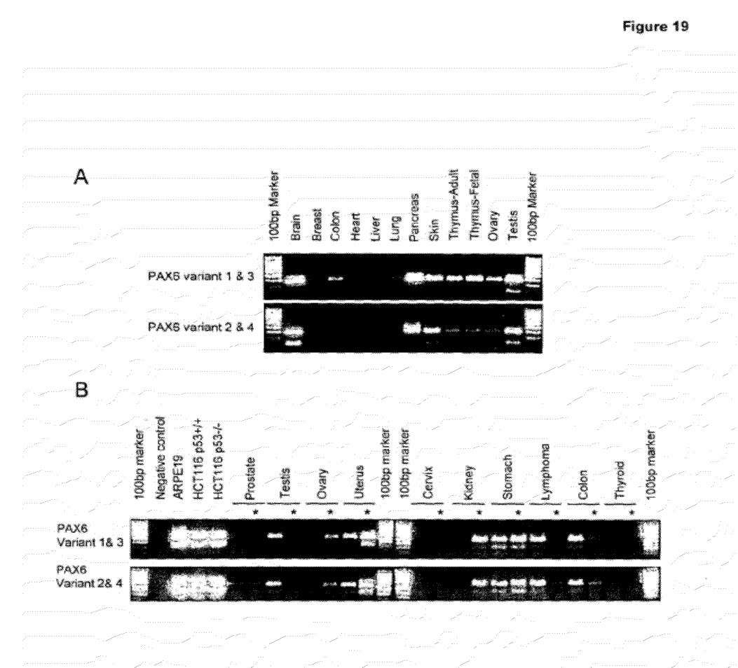

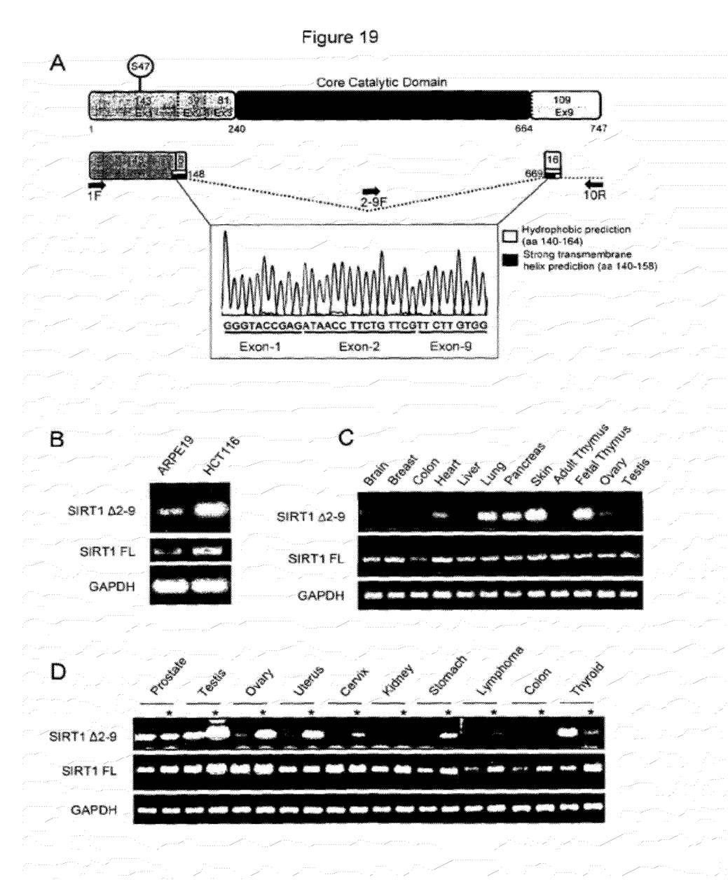

FIG. 19 illustrates identification and tissue expression of a novel alternative splice variant of SIRT1 (A) Identification of a novel alternative splice variant of SIRT1 (SIRT1 Δ2-9). A schematic showing exons of SIRT1 with numbers inside of boxes indicating the number of amino acids in exon, and highlighting serine 47 in exon 1, red color indicates the core catalytic domain (top panel). An unorthodox splicing from within SIRT1 exon-2 to within exon 9 generates SIRT1 Δ2-9; arrows indicate the primers used (middle panel). A sequence trace showing part of SIRT1 exon 1, exon 2 and exon 9 splicing identified in HCT116 cells by 1F/10R primers (lower panel). (B-D) SIRT1 Δ2-9 mRNA expression in ARPE19 (non-cancer retinal epithelial) and HCT116 p53+/+ (colorectal cancer) cell lines (B); in normal human tissues samples (C); and in cancer vs adjacent normal controls (D). Also shown is the expression of SIRT1 FL, and GAPDH as control;

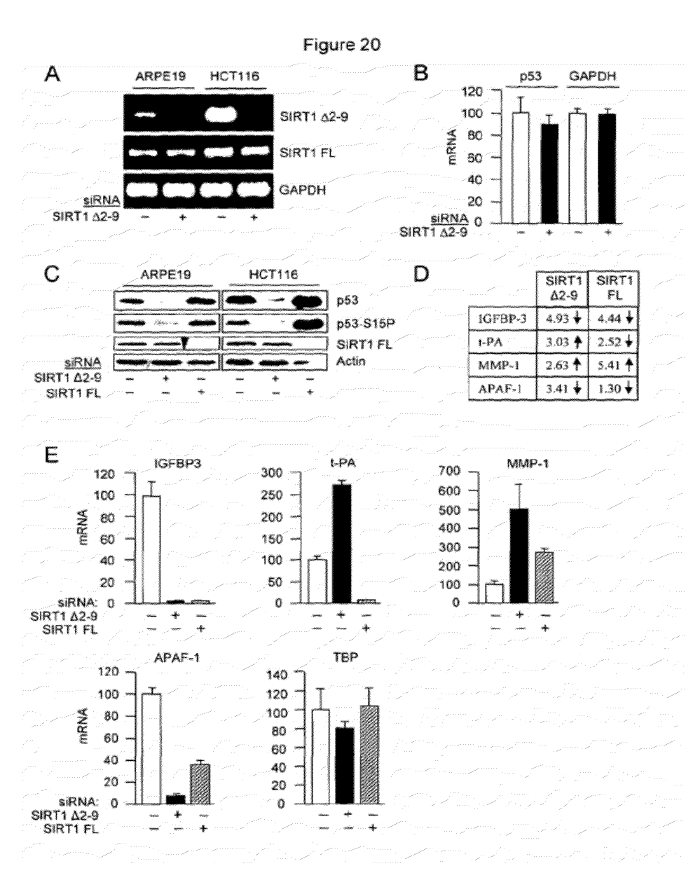

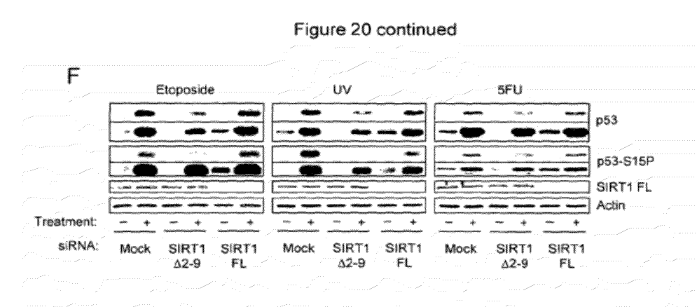

FIG. 20 illustrates SIRT1 Δ2-9 is required for basal p53 protein levels as well as p53 induction in response to stress (A) SIRT1 Δ2-9 RNAi in ARPE19 and HCT116 p53+/+ cell lines. SIRT1 Δ2-9 siRNA specifically depletes SIRT1 Δ2-9 mRNA and has no effects on SIRT1 FL or GAPDH mRNAs. (B) SIRT1 Δ2-9 siRNA does not effect expression of p53 mRNA. SIRT1 Δ2-9 does not effect on the mRNA expression of p53. (C) Ablation of SIRT1 Δ2-9 by RNAi causes depletion of p53 protein in ARPE19 and HCT116 p5c3+/+ cell lines. (D-E) The fold-change values and the direction of regulation of a set of p53-responsive genes identified by SIRT1 Δ2-9 RNAi microarray in ARPE19 cells (D) and verified by qRT-PCR (E). TBP primers were used as control for the RT-PCRs. (F) P53 protein is induced in response to stress but attenuated in response to SIRT1 Δ2-9 siRNA treatment. ARPE19 cells were treated with Etoposide, UV or 5FU alone or in combination with SIRT1 Δ2-9 siRNA and the induction of p53 was measured by western blotting;

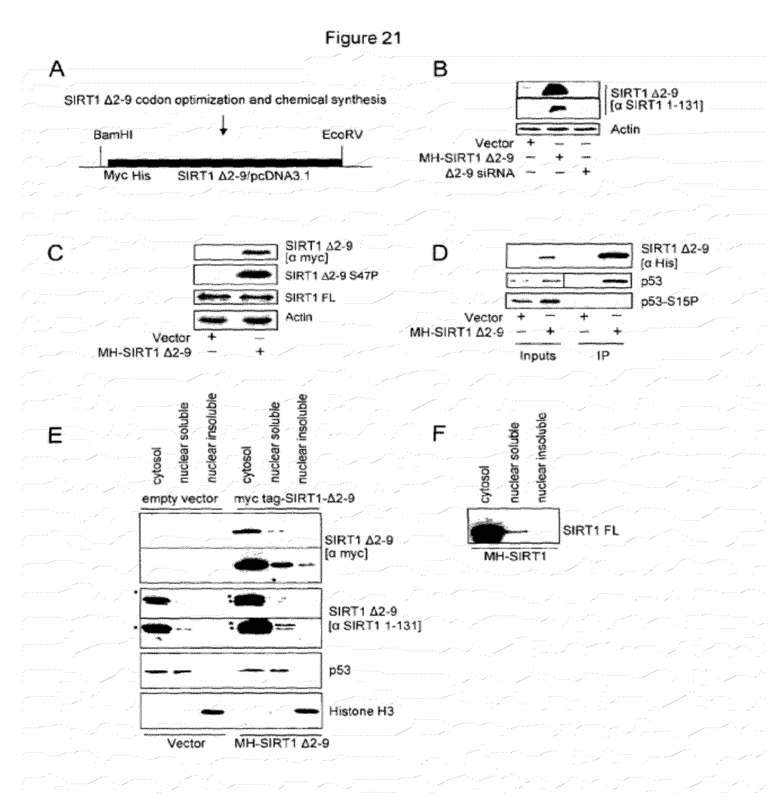

FIG. 21 illustrates SIRT1 Δ2-9 interacts with p53 protein. (A) SIRT1 Δ2-9 expression construct. A schematic showing chemical synthesis and cloning of SIRT1 Δ2-9 in pcDNA3.1 vector for expression in mammalian cells. (B) Expression of SIRT1 Δ2-9 protein. Total cell lysates from HCT116 p53+/+ cells treated with vector alone show endogenous SIRT1 Δ2-9 (lane 1); with Myc-His-SIRT1 Δ2-9 (MH-SIRT1 Δ2-9) show exogenous SIRT1 Δ2-9 protein (lane 2) and SIRT1 Δ2-9 siRNA show ablation of endogenous SIRT1 Δ2-9 protein (lane 3). (C) SIRT1 Δ2-9 is phosphorylated at serine 47. MH-SIRT1 Δ2-9 was exogenously expressed in HCT116 p53+/+ cells and total cell lysates were probed to detect exogenous SIRT1 Δ2-9, SIRT1 Δ2-9-S47P and SIRT1 FL. (D) SIRT1 Δ2-9 interacts with p53. Immunoprecipitation of exogenously expressed MH-SIRT1 Δ2-9 in HCT116 p53+/+ cells co-immunoprecipitates p53 protein (lane 4, cp. Lane 2, inputs). (E) Immunoblots showing the cellular distribution of endogenous and exogenous SIRT1Δ2-9 protein in HCT116 p53+/+ cells following biochemical fractionation into cytosolic, nuclear soluble and nuclear insoluble fractions. Equivalent cell numbers were loaded in each lane and membranes were probed with p53 and histone H3 as controls for successful fractionation. Exogenously expressed SIRT1Δ2-9 detected by anti-myc antibody, longer exposure indicates presence in nuclear insoluble fraction. Endogenous and exogenous SIRT1Δ2-9 were detected with N-terminal SIRT1 antibody; endogenous SIRT1Δ2-9 (·) migrates just below exogenous SIRT1Δ2-9 (*) and shows a similar pattern of fractionation. (F) Immunoblot showing the cellular distribution of SIRT1-FL protein in HCT116 p53+/+ cells. Equivalent cell numbers loaded in each lane; and

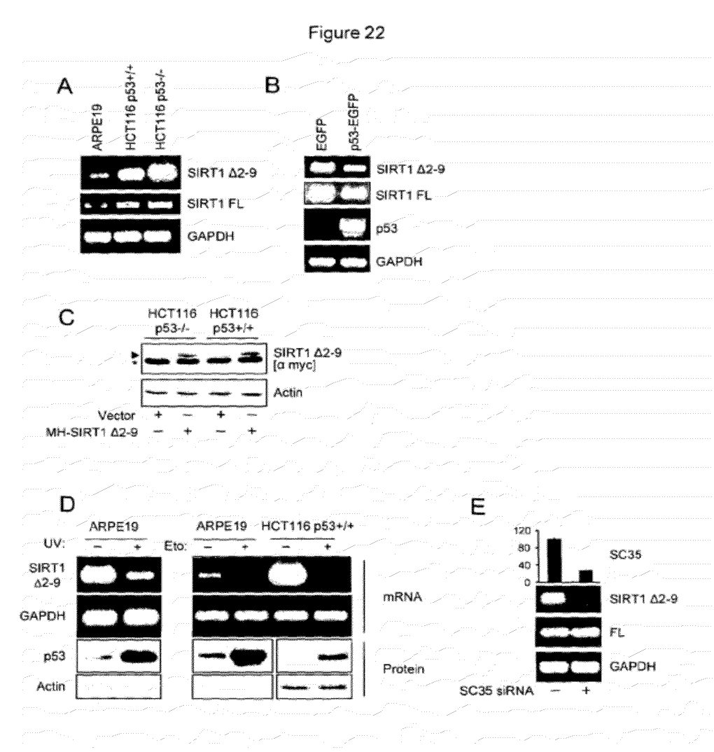

FIG. 22 illustrates the regulation of SIRT1 Δ2-9 transcription. (A) P53 negatively regulates SIRT1 Δ2-9 transcription. Total RNA from ARPE19, HCT116 p53+/+ and HCT116 p53-/- cells were used in RT-PCR to detect transcript levels of SIRT1 Δ2-9, SIRT1 FL and GAPDH. (B) Exogenous expression of p53 in HCT116 p53-/- cells down-regulates SIRT1 Δ2-9 transcript levels.(C) P53 does not effect the expression of exogenous SIRT1 Δ2-9 protein. The level of exogenous SIRT1 Δ2-9 protein expression in HCT116 p53+/+ and HCT116 p53-/- cells is similar. An arrow indicates SIRT1 Δ2-9 protein and * indicates non-specific band. (D) Stress-induced p53 protein down-regulates SIRT1 Δ2-9 transcript. ARPE19 and HCT116 p53+/+ cells were treated with UV and Etoposide. Cell lysates were blotted with p53 and actin antibodies, and RT-PCR for SIRT1 Δ2-9 and GAPDH was performed on the treated and un-treated total RNAs.

(E) SC35 regulates the alternate SIRT1 Δ2-9 splice. Knock-down of SC35 in HCT116 p53+/+ cells via RNAi ablated expression of SIRT1 Δ2-9 mRNA. SIRT1 FL and GAPDH were unaffected;

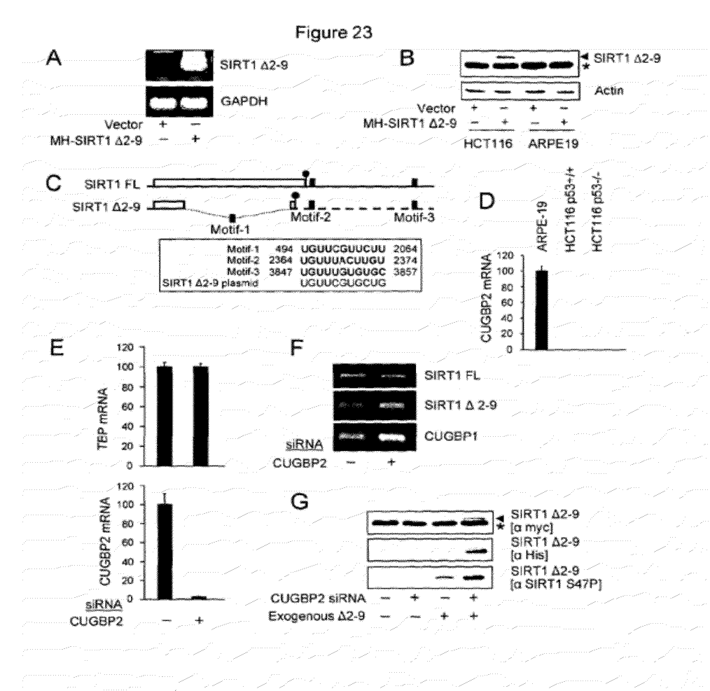

FIG. 23 illustrates that RNA binding protein CUGBP2 negatively regulates SIRT1 Δ2-9 protein expression. (A) Exogenous SIRT1 Δ2-9 mRNA is expressed in HCT116 cells. MH-SIRT1 Δ2-9 mRNA was readily detectable (lane 2) compared to vector transfected only (lane 1). (B) Exogenous SIRT1 Δ2-9 protein is not expressed in ARPE19 cells. Whereas MH-SIRT1 Δ2-9 was readily expressed in HCT116 p53+/+ it did not express in ARPE19 cells. * indicates a non-specific band whereas an arrow indicates SIRT1 Δ2-9 protein. (C) Splicing of SIRT1 exon 2 to exon 9 generates a novel putative CUGBP2 binding motif. A schematic showing putative CUGBP2 binding motifs (orange boxes) in 3′-UTR of SIRT1 FL RNA (solid line). The splicing of part of exon 2 to part of exon 9 creates a putative CUGBP2 binding motif. The RNA sequences of the motifs are shown in the box below. Silent changes were introduced in the putative binding site of CUGBP2 in SIRT1 Δ2-9 by codon optimisation. (D) CUGBP2 mRNA expression. qRT-PCR data shows that CUGBP2 mRNA is not detectable in HCT116 cells. (E-F) CUGBP2 siRNA efficiently silenced CUGBP2 mRNA expression in ARPE19 cells (E) but did not affect SIRT1 Δ2-9 transcript levels (F). (G) Down-regulation of CUGBP2 by siRNA allows the expression of exogenous SIRT1 Δ2-9 protein. Pre-silencing of CUGBP2 prior to introduction of exogenous SIRT1 Δ2-9 permitted a ˜15-fold increase in expression of exogenous SIRT1 Δ2-9; and

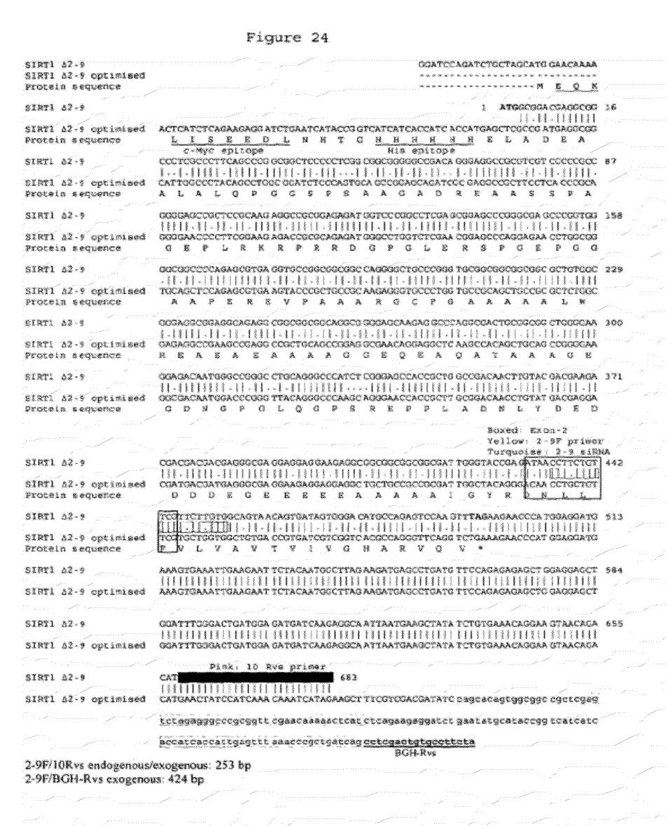

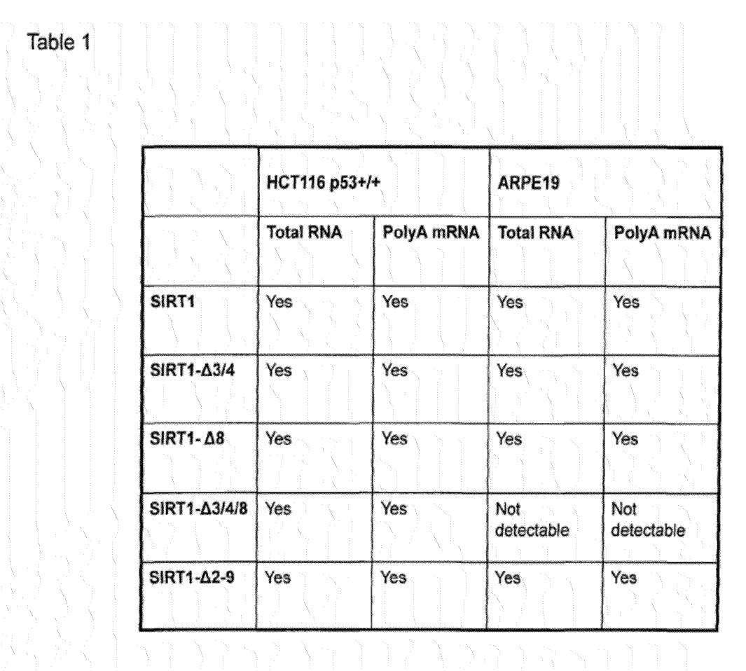

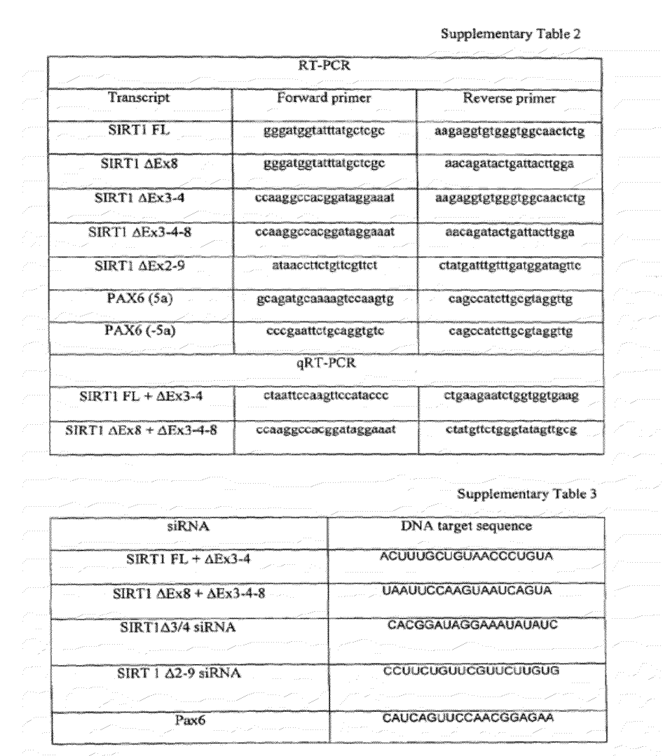

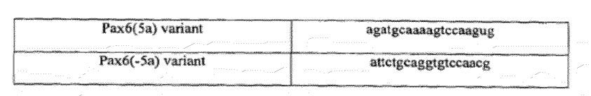

FIG. 24 is the SIRT1 A2-9 Sequence in pcDNA3.1 expression construct. SIRT1 Δ2-9 sequence including its 3′-UTR was codon optimised and chemically synthesised. c-Myc and His epitope tag (underlined) sequences were attached at the 5′-end of SIRT1 Δ2-9 codon optimised sequence, and cloned in pcDNA3.1 vector. The codon optimised sequence is compared with the original nucleotide sequence of SIRT1 Δ2-9, vertical bars indicate identity and dots indicate silent changes introduced for codon optimisation. ATG start and TAG stop codons are shown bold. The primers and siRNA sequences are labelled.

### MATERIALS AND METHODS

Cell Lines and siRNA Transfection

Isogenic colorectal carcinoma cell lines HCT116 p53+/+ and HCT116 p53-/- were cultured in DMEM supplemented with 10% FBS. Non-cancer retinal epithelial cell line ARPE-19 was cultured in DMEM:F12 supplemented with 10% FBS. Cells used in experiments were low passage number (not >8 splits from nitrogen). Transfection of siRNA were as described (Ford et al., 2005; Ford et al., 2008). SIRT1 FL siRNA (targeting SIRT1 FL and SIRT1 ΔEx3-4) is sited within exon8; SIRT1 ΔEx8 siRNA (targeting SIRT1 ΔEx8 and SIRT1 ΔEx3-4-8) is located across the alternate exon7-exon9 splice. Sequences and locations of siRNAs are detailed in the Supplementary Table 3.

Digital phase contrast cell images were captured on an Axiovert Cell Observer (Zeiss). Quantification of apoptosis was performed using an AnnexinV-Fluos kit (Roche) according to the manufacturer's instructions, with analysis by flow cytometry on a FACSCalibur (BD) using CellQuest software.

RT-PCR and qRT-PCR

Standard RT-PCR was performed on a DNA Engine Dyad (MJ Research) using a One-Step RT-PCR kit (Qiagen). Reaction endpoints were electrophoresed on agarose gels, visualised on a UV transilluminator (Appligene) and images captured with a DS34 camera (Polaroid). Non-cancer tissue RNA samples and paired non-cancer/cancer tissue RNA samples were obtained from AMS Biotechnology Europe and Ambion respectively. Total RNA from cell experiments was isolated using an RNeasy kit (Qiagen) and quantitated by UV spectroscopy (GeneSpecV). For qRT-PCR, reactions were run in quadruplicate on a DNA Engine Opticon (BioRad) using a QuantiTect SYBR Green RT-PCR kit (Qiagen). Sequences of the primers used in RT-PCR and qRT-PCR are given in supplementary table 2; a schematic of the location of the primers is given in FIG. 8. The general cycling conditions were: 50° C. for 30 minutes, 94° C. for 15 minutes followed by the thermal cycle 94° C. for 10 seconds, annealing for 30 seconds, and 72° C. extension for 30 seconds repeated for a number of cycles specific for each primer pair. The specific variations were as follows. In standard RT-PCR, SIRT1 FL was annealed at 58° C. and cycle repeated for 34 times; SIRT1 ΔEx3-4 and SIRT1 ΔEx8 were annealed at 53° C. and cycle repeated for 34 times; SIRT1 ΔEx3-4-8 and SIRT1 ΔEx2-9 were annealed at 53° C. and cycle repeated for 44 times.

In qRT-PCR, SIRT1 FL was annealed at 55° C., and the cycle repeated 34 times. SIRT1 ΔEx8 was annealed at 50° C. and the cycle repeated 43 times. The primers and cycling condition for GAPDH housekeeper control have been described (Ford et al., 2005).

### Fenretinide Treatment of ARPE19 Cells

Cells were treated for 48 h with 1 μM fenretinide in OPTIMEM media 24 h following siRNA transfection. Before addition of fenretinide cells were first washed with OPTIMEM to remove serum. 48 h later fenretinide was removed by washing with OPTIMEM and normal growth media with serum was added. Cells were then monitored by microscopy for a further 48 h.

### Example 1

### Identification of Additional SIRT1 Splice Variants

In our present study a more detailed characterisation of SIRT1 RNA expression has revealed five major SIRT1 transcripts expressed in human cells. Each transcript was cloned and sequenced up-stream and down-stream to confirm the splice junctions (Methods; FIG. 7). In addition to SIRT1-FL we confirmed the presence of SIRT1-Δ8 which is generated by precise splicing between exons 7 and 9 and results in an in-frame mRNA product. A second splicing event removes SIRT1 exons 3 and 4 to generate

SIRT1-Δ3/4 and SIRT1-Δ3/4/8. In both cases the reading frame at the start of exon 5 is shifted (FIG. 1A, 7C,D) and identical truncated protein products for each transcript are predicted due to a premature stop codon in exon 5 (FIGS. 1A, 10, 11). The fourth SIRT1 splice variant is spliced from within exon 2 to within exon 9 and contains a premature stop codon within exon 9 (SIRT1 Δ2-9; FIGS. 1A, 12). All SIRT1 splice variants are polyadenylated (Supplementary Table 1).

Variant-specific RNA primers were designed in order to compare basal expression levels of the individual SIRT1 transcripts in different cell lines and in different human tissues (Methods; Supplementary Table 2). Cell lines included immortalised, partially differentiated human retinal epithelial cells (ARPE19), and two isogenic clones of HCT116 human colorectal cancer cells (HCT116 p53+/+ and HCT116 p53-/-). SIRT1 FL, SIRT1-Δ8 and SIRT1-Δ3/4 were expressed in all three cell lines. Higher levels of SIRT1-FL and SIRT1-Δ8 were expressed in HCT116 cells compared with ARPE19 cells (Cian Ref and FIG. 1B). SIRT Δ8 was most highly expressed in HCT116 p53-/- cancer cells (FIG. 1B), consistent with the negative feed-back loop previously observed between p53 and SIRT1 Δ8 . The variant SIRT1 Δ3/4/8 was not expressed in ARPE19 cells. SIRT1-Δ3/4/8 was very highly expressed in HCT116 p53-/- cells relative to HCT116 p53+/+ cells indicating that p53 may suppress the generation of SIRT1 Δ3/4/8 in the isogenic HCT116 p53+/+ cells (FIG. 1B). In contrast the splice variant SIRT1 Δ2-9 was expressed at equally high levels in both HCT116 p53+/+ and HCT116 p53-/- cells, suggesting that SIRT1 Δ2-9 splicing is p53-independent (FIG. 1B). Interestingly however,

SIRT1 Δ2/9 was barely detectable in non-cancer ARPE19 cells (FIG. 1B). This raises the possibility that SIRT1 Δ2-9 may be associated with malignant transformation of human cells (see also comparison of paired cancer and non-cancer tissues, below).

### Example 2

### SIRT1 Variant RNAs are Differentially Expressed in Human Tissues

SIRT1 FL was expressed at similar levels in a range of human tissues, with the exception of colon which contained a relatively lower level of SIRT1 FL transcript (FIG. 1C; see also REF). The colon also expressed low levels of SIRT1 Δ8, as did liver and adult thymus (FIG. 1C). Interestingly foetal thymus expressed much higher levels of SIRT1 Δ8, indicating that SIRT1 Δ8 may have a developmental role in the thymus.

SIRT1 Δ3/4 was evident in all tissues tested but with varying levels (FIG. 1C). In contrast SIRT1 splice variants SIRT1 Δ3/4/8 and SIRT1 Δ2-9 exhibited tissue-specific expression. For example, SIRT1 Δ3/4/8 was readily detectable in foetal thymus, the testis and ovarian tissues, but was low or non-detectable in a range of other tissues tested including adult thymus (FIG. 1C). The variant SIRT1 Δ2-9 was predominant in foetal thymus, lung, prostate and stomach (FIG. 1C). SIRT1 Δ2-9 was not detectable in adult thymus. The differential expression of SIRT1 variant transcripts in human tissues may be functionally important or may simply reflect aberrant splicing prevalencies in different tissue types. In the thymus SIRT1 splicing may be particularly significant since both adult and foetal thymus express similar levels of SIRT1 FL whereas foetal thymus expresses much higher levels of all three SIRT1 splice variants compared with adult tissue (FIG. 1C).

### Example 3

### Comparison of Paired Human Cancer and Non-Cancer Tissues

A range of paired tissue samples of cancerous and adjacent non-cancerous origin were also screened for expression of SIRT1 RNA splice variants. The most striking differences between cancer and non-cancer tissues were observed for SIRT1 Δ3/418 and SIRT1 Δ2/9 (FIG. 2). In the case of SIRT1 Δ3/4/8 high RNA expression levels were observed in normal testis and cervix but SIRT1 Δ3/4/8 RNA was undetectable in the adjacent cancerous tissue. Conversely in the stomach SIRT1 Δ3/4/8 was undetectable in normal tissues but was highly expressed in adjacent cancerous tissue (FIG. 2). Thus expression of the SIRT1 Δ3/4/8 variant appears to be unpredictable and this is further re-enforced by marked differences in its expression in two individual samples of non-cancerous ovarian tissue (FIG. 1C, ovary: SIRT1 Δ3/4/8-positive cp FIG. 2, ovary: SIRT1 Δ3/4/8-negative).

The expression of SIRT1 Δ2/9 was detectable in both human cancer and paired non-cancer tissues but, importantly, exhibited cancer-related increases in the testis, the ovary, the uterus, the stomach and cervix (FIG. 1D). This is consistent with high expression of SIRT Δ2/9 in human cancer cell lines compared with non-cancer cells (see above) and re-enforces the possibility that SIRT1 Δ2/9 may in some way be linked with initiation and/or progression of human cancer.

### Example 4

### SIRT1 Splicing Involves Splicing Factors MTR4 and SC35

The SR splicing factor SC35 has been identified as important for splicing of SIRT1 FL in both murine and human cells (MCB; Cian). Using RNAi we confirm this observation for human HCT116 cancer and ARPE19 non-cancer cells (FIG. 14). Levels of SIRT1-FL were reduced following silencing of SC35 in HCT116 cancer cells and in ARPE19 non-cancer cells. SIRT1-Δ8 was also reduced in SC35-depleted ARPE19 cells. However SIRT1 Δ8 expression was unaffected by SC35-depletion in HCT116 cells (FIG. 14) raising the possibility of mechanistic differences in the processing of SIRT1 RNA in cancer versus non-cancer cells. This was further supported by cancer-related differences in SIRT1 Δ3/4 expression which increased some 3- to 4-fold following SC35 silencing in HCT116 cancer cells but increased to a much lesser extent in SC35-silenced ARPE19 non-cancer cells . Overall these results indicate that SC35 directs SIRT1 RNA splicing to generate the full length SIRT1 mRNA and also SIRT1 Δ8 RNA. Splicing between exons 2 and 5 is apparently suppressed in the presence of SC35 in HCT116 cancer cells since 43,4 variants increase following SC35 silencing. This effect is independent of p53 (Fig., SIRT1 Δ3,4 and Δ3/4/8, compare HCT116 p53+/+ and p53-/-).

We also tested the effects of MTR4 on SIRT1 RNA processing. MTR4 is a member of the DEAD-box family of ATP-dependent helicases and a co-factor of the exosome complex and is linked with RNA surveillance and quality control . Selective knock-down of MTR4 by RNAi caused 60-80% decrease in both SIRT1-FL RNA and SIRT1-Δ8 in HCT116 p53+/+ and ARPE19 cells (FIG. 14). Interestingly, HCT116 p53-/- cells appeared to be refractory to MTR4 silencing, indicating that MTR4 involvement in SIRT1 RNA processing is p53-dependent. Expression levels of SIRT1 Δ3/4/8 RNA were unaffected by MTR4 silencing in all three cell lines.

These combined observations (indicate that the pattern of SIRT1 RNA splicing is independently influenced by SC35 and MTR4 and is variable between cancer and non-cancer cells. In addition the tumour suppressor protein p53 also seems important since the involvement of MTR4 is lost in p53-/- HCT116 cells (see above). RNAi depletion of MTR4 was performed in the absence of applied stress, and under conditions that fail to activate a p53 stress response. We therefore conclude that p53 constitutively favours MTR4-dependent production of both SIRT1-FL and SIRT1 Δ8 RNA under basal conditions of cell growth. The observed link between MTR4 and p53 is, to our knowledge, novel and may have more general impact upon RNA integrity in p53-deficient cells. Future studies will explore this possibility since loss of p53 function is linked with most human cancers.

### Example 5

### Cloning and Expression of SIRT1 Δ8 Protein

The splicing of SIRT1 Δ8 generates an in-frame SIRT1 RNA product (FIG. 1, 9). To ask if SIRT1 Δ8 is expressed as a protein SIRT1 Δ8 RNA was cloned and engineered into a mammalian expression vector (FIG. 15A). Expression was driven by the CMV promoter and the expressed protein was tagged at the C-terminus with c-Myc and 6× His for ease of detection. A 95 kDa protein product reactive with anti-c-Myc antibody was detectable following transient transfection in both ARPE19 and HCT116 cells (FIG. 15B for HCT116). This product was also detectable with anti-SIRT1 antibodies raised against an N-terminal SIRT1 peptide 1-131 and C-terminal SIRT1 peptide 448-747 (FIG. 15B). We conclude that SIRT1 Δ8 is translated in-frame into SIRT1 Δ8 variant protein. SIRT1-Δ8 lacks residues 452-637 encoded by SIRT1 exon 8, thus refining the epitope target region for the H300 anti-SIRT1 polyclonal antibody (Santa Cruz) to residues 638-747 of SIRT1 protein. We further demonstrate that SIRT1 Δ8 is phosphorylated at serines 27 and 47 (S27P and S47P; FIG. 15B). From this we infer that the SIRT1 kinase(s) recognise and phosphorylate the exogenously expressed SIRT1 Δ8 protein.

### Example 6

Effects of SIRT FL and SIRT1 Δ8 upon p53 Acetylation

SIRT1 down-regulates the acetylation status of the tumour suppressor protein p53 (i) by de-acetylating p53 directly, and (ii) by de-acetylating the acetyl transferase p300 which is responsible for acetylation of p53. SIRT1-catalysed deacetylation of p300 down-regulates p300 activity. Direct and/or indirect down-regulation of p53 acetylation by SIRT1 is predicted to have a profound impact upon the ability of cells to withstand stress since acetylation is essential for the p53 stress response.

Importantly, SIRT1 also impacts upon the balance of p53 acetylation/de-acetylation under basal non-stress conditions. This is evident from the massive increase in p53 acetylation following selective silencing of SIRT1 FL in HCT116 cells using siRNAs directed against exon 8 of SIRT1 mRNA. Here we confirm and extend this observation and demonstrate that p53 acetylation levels increase more than 50-fold following SIRT1 FL depletion under basal conditions in HCT116 cells (FIG. 16A) whilst total p53 protein accumulates approximately 10-fold. This indicates (i) that p53 is continually being acetylated/de-acetylated in HCT116 cells under basal conditions of cell culture and (ii) that the presence of SIRT1 FL constitutively maintains p53 in its hypo-acetylated state.

Assuming that acetylation is attributable to p300, this also indicates that SIRT1 FL can selectively de-acetylate p53 without at the same time de-acetylating p300, thus accounting constitutive p53 acetylation and the accumulation of acetylated p53 following SIRT1 silencing. These results also suggest that SIRT1, directly or indirectly, constitutively suppresses p53 protein levels under basal conditions in HCT116 epithelial cancer cells. Since p53 protein turnover is subject to complex regulation (Refs) further studies are necessary to elucidate this previously undescribed effect of SIRT1.

Compared with SIRT FL the depletion of SIRT1 Δ8 (under basal conditions) only had a minimal effect upon p53 acetylation status. Nonetheless a small reproducible increase of 2 to 3-fold p53 acetylation was observed (FIG. 16A). This was evident in both HCT116 cancer cells and in ARPE19 non-cancer cells (FIG. 16A,B). In addition SIRT1 Δ8 appeared to down-regulate total p53 protein levels in ARPE19 cells since selective depletion of SIRT1 Δ8 caused a 2 to 3-fold increase in p53 protein (FIG. 16B). A similar effect was not observed in HCT116 cells (FIG. 16A). The effects of combined silencing of SIRT1 FL plus SIRT1 Δ8 in ARPE19 were additive (FIG. 16A,B). These results were re-enforced by the observation that over-expression of exogenous SIRT1 Δ8 caused a small but reproducible decrease in p53 protein (FIG. 16C) indicating that SIRT1 Δ8 influences cellular levels of p53 protein under basal, non-stress conditions, and also impacts upon p53 protein acetylation. Reciprocal regulation between SIRT1 Δ8 and p53 thus contributes to a negative feed-back loop operating between these two crucial regulators of cellular hoemostasis.

### Example 7

SIRT1 Δ8 Forms Protein-Protein Complexes with p53 and AROS

The observation that SIRT1 Δ8 in some way influences p53 protein levels and acetylation status, albeit to much lower levels than SIRT1 FL, suggests that SIRT1 Δ8 may physically interact with p53 protein. To investigate this we performed a series of pull-down assays and examined SIRT1 Δ8 binding capacity for p53, and also for other established SIRT1-binding proteins AROS and DBC. The results show that exogenous SIRT1 Δ8 binds p53 and AROS proteins (FIG. 17A,B). However, no evidence of complexes between SIRT1 Δ8 and DBC, a negative regulator of SIRT1 deacetylase activity was observed (FIG. 17A). In addition we failed to find any evidence for SIRT1-FL/SIRT1-Δ8 complexes (FIG. 17C).

### Example 8

### Sub-Cellular Localisation of SIRT1 Δ8

The sub-cellular localisation of SIRT1 Δ8 was compared with SIRT1 FL by cell fractionation. Both SIRT1 FL and SIRT1 Δ8 localised in the cytosolic fraction and to a lesser extent in the soluble nuclear fraction (FIG. 18A). A faint band of SIRT1 Δ8 was also observed in the nuclear-bound fraction, indicating that SIRT1 Δ8 may bind to nuclear sub-structures and/or chromatin.

We have previously demonstrated that RNAi-mediated silencing of JNK2 (but not JNK1) reduces SIRT1 FL protein due to reduced protein stability (REF). Here we show a similar effect for SIRT1 Δ8 and further demonstrate that both full length and SIRT1 Δ8 are selectively lost from the soluble nuclear fraction following JNK2 depletion (FIG. 18B).

### Example 9

### SIRT1 Δ8 has a Short Half-Life

Although exogenous SIRT1 Δ8 was readily detectable by immunoblotting of cell lysates and was reactive with anti-SIRT1 antibodies (see above), the detection of the endogenous SIRT1 Δ8 protein was difficult. One possible explanation is that SIRT1 Δ8 may have a shorter half life than SIRT1 FL. This was investigated by time course analyses following inhibition of cellular protein synthesis with cycloheximide (CHX; FIG. 18C-H). The results demonstrate a short half-life of around 2 hr for SIRT1 Δ8 compared with >9 hr for SIRT1 FL (FIG. 18C,D).

The half-life of SIRT1 Δ8 in HCT116 cells was prolonged following RNAi-mediated silencing of p53, and shortened following RNAi depletion of JNK2 (FIG. 18E-G). This indicates that in some way the presence of p53 shortens, whilst JNK2 prolongs SIRT1 Δ8 protein stability in HCT116 cells. Similar results have been previously observed for the stability of SIRT1 FL protein (REF).

In ARPE19 cells RNAi-mediated depletion of p53 had minimal effect upon SIRT1 FL protein levels. However p53 depletion induced a dramatic increase in SIRT Δ8 protein levels and this was paralleled by increased S27P (FIG. 18H). The short half-life of SIRT1 Δ8 compared with SIRT1 FL suggests that the protein domain encoded by SIRT1 exon 8 is linked with SIRT1 protein stability. In addition S27P correlates with both SIRT FL and SIRT Δ8 protein turnover (see above and Ref.) We conclude (i) that the half lives of both SIRT1 FL and SIRT Δ8 are linked with SIRT1 S27 phosphorylation, (ii) that the presence of p53 can selectively affect SIRT1 Δ8 protein stability, and (iii) that the “exon 8 domain” of SIRT1 protein helps stabilise SIRT1 FL under basal conditions of cell growth.

### Example 10

### SIRT1 Δ8 and Cancer Cell Survival

Using siRNAs targeted against exon 8 of SIRT1 mRNA we have previously identified SIRT1 (FL) as a cancer-specific survival factor (Ref×2). To ask if SIRT1 Δ8 also influences cell survival we next employed a siRNA targetting the exon 7/9 junction of SIRT1 mRNA to selectively target SIRT1 D8 mRNA (siRNA sequence shown in Supplementary Table 3). In HCT116 colorectal cancer cells the silencing of SIRT1 FL induced massive apoptosis (FIG. 3). The effect was p53-independent, although apoptosis was delayed by approximately 24 hr in HCT116 p53-/- cells (FIG. 3B. see also Ref). In contrast the selective silencing of SIRT1 Δ8 had little or no apparent effect on HCT116 cells and did not induce apoptosis of these cancer cells (FIG. 3). It remained possible that SIRT1 Δ8 may influence apoptosis via a pro-apoptotic function promotes cancer cell death following silencing of SIRT1 FL. In this context it is relevant to note that a similar relationship is evident for JNK1 and JNK2 whereby JNK2 suppresses JNK1-mediated apoptosis and co-silencing JNK1 with JNK2 rescues JNK2-depleted HCT116 cells from apoptosis . However a similar relationship between SIRT1 FL and SIRT1 Δ8 was not observed in the present study since co-silencing SIRT1 FL plus SIRT1 Δ8 failed to rescue HCT116 cells from apoptosis (FIG. 3). We conclude that SIRT1 Δ8 is not an essential pro-apoptotic mediator under these conditions. Similar results were observed for both HCT116 p53+/+ and HCT116 p53-/- cells (FIG. 3B)). We conclude that the functions of SIRT1 FL and SIRT1 Δ8 in relation to the regulation of cancer-specific apoptosis are distinct and that SIRT1 Δ8 is dispensable for HCT116 cancer cell survival whilst SIRT-FL is essential for the survival of these same cells under basal (non-stress) conditions of growth.

### Example 11

### SIRT1 FL Silencing Induces Neuronal Differentiation in ARPE19 Cells

In contrast to HCT116 epithelial cancer cells the survival of ARPE19 non-cancer epithelial cells appears to be independent of SIRT1-FL since selective RNAi-mediated silencing of SIRT1 FL fails to induce apoptosis (REFs; see also FIG. 4A). In all our RNAi experiments the cells are transfected with a single dose of siRNA and observed at various times thereafter, unless otherwise stated (Methods). At 48 h the morphological phenotype of SIRT1 FL-depleted ARPE19 cells is relatively unchanged whereas HCT116 cells are apoptotic (confirmed by annexin V staining; see above and FIG. 3). Interestingly, after 72 h to 96 hr the morphology of selectively-depleted SIRT1-FL ARPE19 cells undergoes striking changes, reminiscent of differentiation towards the neuronal phenotype (FIG. 4A). In this context it should be noted that ARPE19 cells are partially differentiated retinal pigmented epithelial cells derived from non-cancerous tissue. ARPE19 have a normal karyotype and retain many of the structural and physiological properties of normal retinal pigmented epithelial cells in vivo (Dunn et al., 1996), including polarised membrane expression of monocarboxylate transporters (Philp et al., 2003). Moreover, ARPE19 cells are programmed to undergo neuronal differentiation which can be induced extrinsically by fenretinide, a retinoic acid derivative (FIG. 6). Unexpectedly, our present results indicate that selective depletion of SIRT-FL also induces neuronal differentiation of ARPE19 cells. From this we infer that SIRT-FL is a constitutive and intrinsic suppressor of neuronal differentiation in ARPE19 cells.

### Example 12

### Opposing Effects of SIRT-FL and SIRT1 Δ8 on Differentiation of ARPE19 Cells

The selective silencing of SIRT1 Δ8, in contrast to SIRT1-FL silencing, did not induce morphological differentiation of ARPE19 cells towards the neuronal phenotype (FIG. 4A). However, co-silencing SIRT1 Δ8 plus SIRT1 FL abolished the effects of SIRT1 FL silencing alone (FIG. 4) indicating that SIRT1 Δ8 is required for neuronal differentiation of ARPE19 cells. To explore the influence of SIRT1 on neuronal differentiation in more detail we also co-silenced SIRT1-FL with SIRT1 Δ3/4. With this silencing combination the morphological differentiation towards the neuronal ARPE19 phenotype in response to SIRT1-FL silencing was unaffected (FIG. 4A). Thus the SIRT1 requirement for neuronal differentiation is selective for SIRT1-Δ8. We conclude that SIRT1 FL and SIRT1 Δ8 exert opposing effects upon the intrinsic programming of neuronal differentiation in ARPE19 retinal epithelial cells.

### Example 13

### Stem Cell Factor PAX6 is Required for SIRT1-Regulation of Neuronal Differentiation

PAX6 is a stem cell factor with critical roles in the development of mammalian brain, eye and pancreas (Refs). SIRT1 is also implicated in the development of these same organs in mammals (Refs). Given our discovery that SIRT1 FL and SIRT Δ8 differentially regulate neuronal differentiation of ARPE retinal epithelial cells we next asked if PAX6 is linked with the functioning of these newly identified SIRT1 variants in ARPE19 cells.

First we show that PAX6 mRNA is expressed at high levels in human tissues of the brain, pancreas, colon, skin, thymus ovary and testis (FIG. 19A). Pax6 is also highly expressed in ARPE19 cells (FIG. 19B). This is expected since ARPE19 cells are neuronal in origin. PAX6 siRNA gave good PAX6 mRNA knock-down but did not affect the general morphological phenotype of ARPE19 cells under basal conditions of growth (FIG. 5A). Similarly there was no apparent effect induced by co-silencing PAX6 plus SIRT1 Δ8. However, co-silencing PAX6 plus SIRT1 FL abolished the morphological differentiation induced by selective silencing of SIRT1 FL alone (FIG. 5A). This indicates (i) that PAX6 is required, together with SIRT1 Δ8, for ARPE19 cell differentiation towards the neuronal phenotype, and (ii) that both SIRT1 Δ8 and PAX6 are subject to regulation by SIRT1-FL which can block neuronal differentiation

### Example 14

### Identification and Tissue Expression of a Novel Alternative Splice Variant of SIRT1 SIRT1 Δ2-9

During the process of cloning SIRT1 FL by RT-PCR from HCT116 cells by using 1F/10R primers which amplify full-length SIRT1 (FIG. 19A), other prominent DNA bands were observed. These PCR products without gel purification were used in ligation reactions and subsequent cloning steps. Further analysis of the recombinant colonies revealed several 0.8 Kbp inserts. These 0.8 Kbp clones were subsequently sequenced to reveal that all these clones contain SIRT1 sequence that is alternatively spliced in an unorthodox splicing from within exon-2 to within exon-9. This alternate transcript of SIRT1 was named as SIRT1 Δ2-9 (FIG. 19A). SIRT1 Δ2-9 transcript is 736 by encoding a protein of 164 amino acids. Most of the SIRT1 Δ2-9 sequence is coded in exon 1, exon 2 codes for only 5 amino acids. The splicing of exon-2 to exon-9 shifts the reading frame which results in the addition of premature stop codon. SIRT1 Δ2-9 variant was polyadenylated (data not shown). SIRT1 2-9 retains coding sequence of exon-1 and part of exon-2 as well as a novel stretch of 16 residues from exon-9 therefore, it lacks the central core catalytic domain, NAD and substrate binding sites. It also lacks the sequences which are involved in binding to DBC-1, whereas AROS binding domain is partially retained. SIRT1 Δ2-9 however retains one of the Nuclear Localisation and Nuclear Export Signals, together with serine 27 and serine 47 phosphorylation sites. SIRT1 Δ2-9 mRNA is expressed at low levels in ARPE19 retinal epithelial cells and over-expressed in colon cancer cell line HCT116 (FIG. 19B). Analysis of SIRT1 Δ2-9 mRNA expression in a panel of normal human tissues showed that SIRT1 Δ2-9 mRNA is expressed in most of the tissues with the highest expression observed in fetal thymus, skin, lung, and pancreas (FIG. 19C). Scanning of tumor vs normal adjacent control tissues identified that SIRT1 Δ2-9 is up-regulated in 6 out of 10 tumor samples tested (FIG. 19D).

### Example 15

SIRT1 Δ2-9 is Required for Basal p53 Protein Levels as well as p53 Induction in Response to Stress

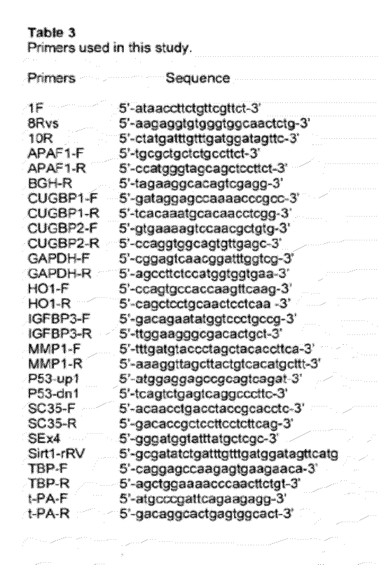

In order to look at the functional significance of SIRT1 Δ2-9, a siRNA was designed to the splice junction region of exon-2 and exon-9. ARPE19 and HCT116 cells were transfected with SIRT1 Δ2-9 siRNA. FIG. 20A shows that SIRT1 Δ2-9 siRNA almost completely abolished SIRT1 Δ2-9 mRNA expression. Western blot analysis of ARPE19 and HCT116 p53+/+ cells treated with SIRT1 Δ2-9 siRNA showed that p53 protein is significantly reduced in response to SIRT1 Δ2-9 siRNA in both ARPE19 and HCT116 p53+/+ cells (FIG. 20C). P53-serine 15 phosphorylation was also reduced (FIG. 20C). This effect was only at the p53 protein levels and not at the p53 transcript levels (FIG. 208). SIRT1 Δ2-9 did not have a significant effect on SIRT1 FL protein levels (FIG. 20C). FIG. 20D shows the regulation and fold-changes of transcript levels of p53-regulated genes identified by SIRT1 Δ2-9 RNAi microarray in ARPE19 cells. These changes were also confirmed by quantitative RT-PCR (FIG. 20E). As SIRT1 Δ2-9 is shown to be required for basal p53 protein stability, we next asked whether it is also required for p53 induction in response to stress. ARPE19 cells were treated with different DNA damaging agents, SFU, Etoposide and UV. P53 is induced substantially in response to the treatments (FIG. 20F), but attenuated in SIRT1 Δ2-9 siRNA treated samples (FIG. 20F). Stress or SIRT1 Δ2-9 siRNA treatment did not have any effect on SIRT1 protein levels (FIG. 20F). These results indicate that SIRT1 Δ2-9 is required for maintaining both the basal levels of p53 protein as well as p53 induction in response to stress.

### Example 16

SIRT1 Δ2-9 protein interacts with p53

Due to the very high GC content, SIRT1 Δ2-9 was difficult to clone, therefore the DNA sequence of SIRT1 Δ2-9 was codon optimised and cloned in pcDNA3.1 vector with Myc and His epitope tags at its 5′ end (FIG. 21A). Cells were transfected with SIRT1 Δ2-9 construct or SIRT1 Δ2-9 siRNA. Western blot analysis with SIRT1 N-terminus antibody showed that both exogenous and endogenous SIRT1 Δ2-9 proteins are detectable in HCT116 cells (FIG. 21B). Further analysis of the exogenous SIRT1 Δ2-9 protein expression showed that SIRT1 Δ2-9 serine 47 is phosphorylated and the over-expressed SIRT1 Δ2-9 has no effect on SIRT1 FL protein levels (FIG. 21C). Exogenous SIRT1 Δ2-9 protein was immunoprecipitated with His antibody and blotted with p53 antibody. The result shows that SIRT1 Δ2-9 physically interacts with p53 (FIG. 21D). It is interesting to note that this interaction excludes the serine 15 phosphorylated form of p53 (FIG. 21D, lower panel). The results in FIG. 3E show that most of the SIRT1 Δ2-9 protein is cytosolic with some protein detectable in nuclear soluble and insoluble fractions, and this pattern of SIRT1 Δ2-9 distribution is similar to SIRT1 FL (FIG. 21F).

### Example 17

Regulation SIRT1 Δ2-9 transcription

The first indication that p53 might be negatively regulating SIRT1 Δ2-9 transcription came from the analysis of SIRT1 Δ2-9 transcript levels in HCT116 p53+/+ and HCT116 p53-/- cells. As shown in FIG. 22A, SIRT1 Δ2-9 is highly expressed in HCT116 p53-/- cells compared to HCT116 p53+/+ cells. We envisaged that if this is the case then over-expressing p53 in HCT116 p53-/- cells should reduce SIRT1 Δ2-9 mRNA expression, and indeed SIRT1 Δ2-9 expression is reduced in HCT116 p53-/- cells where p53 is ectopically expressed (FIG. 22B), FIG. 4C shows that p53 does not effect the expression of exogenous SIRT1 Δ2-9 protein. We next asked if stress induced p53 would affect SIRT1 Δ2-9 transcript levels. ARPE19 and HCT116 cells were treated with UV, Eto and 5FU which mechanistically induce DNA damage in different ways. The treatments of cells with these stresses induce p53 protien levels (FIG. 22D lower panel) and a concomitant reduction in SIRT1 Δ2-9 transcript levels (FIG. 22D, upper panel). In summary, these experiments indicate that p53 negatively regulates SIRT1 Δ2-9 transcription, FIG. 22E shows that splicing factor SC35 regulates SIRT1 Δ2-9 splicing.

### Example 18

### RNA Binding Protein CUGBP2 Negatively Regulates SIRT1 Δ2-9 Protein Expression

The exogenous SIRT1 Δ2-9 mRNA is abundantly expressed in ARPE19 cells (FIG. 23A), but the exogenous protein was barely detectable, whereas it is readily detectable in HCT116 cells (FIG. 23B). We noticed that SIRT1 3′-UTR sequence contains 2 putative CUGBP2 binding motifs and the splicing of SIRT1 exon-2 to exon-9 generates another novel putative CUGBP2 binding motif (FIG. 23C). CUGBP2 is an RNA binding protein involved in splicing as well as translational control. CUGBP2 mRNA is highly expressed in ARPE19 cells but barely detectable in HCT116 cells (FIG. 23D). We reasoned that CUGBP2 may be regulating the translation of SIRT1 Δ2-9 in ARPE19 cells. A siRNA was designed which specifically silences CUGBP2 mRNA expression in ARPE19 cells (FIG. 23E), but did not effect the endogenous levels of SIRT1 FL or SIRT1 Δ2-9 mRNA (FIG. 23F) suggesting that CUGBP2 is not regulating SIRT1 Δ2-9 splicing. We next silenced CUGBP2 alone or in combination with the over-expression of exogenous SIRT1 Δ2-9. As shown in FIG. 23G the silencing of CUGBP2 followed by over-expression of exogenous SIRT1 Δ2-9 allowed the detection of exogenous SIRT1 Δ2-9 protein suggesting that CUGBP2 is negatively regulating SIRT1 Δ2-9 translation possibly through binding to the putative CUGBP2 binding motif generated by splicing of exon-2 to exon-9 (as the other two motifs in the 3′-UTR were absent in the exogenous SIRT1 Δ2-9 construct). We also show that CUGBP2 protein binds to both endogenous and exogenous SIRT1 Δ2-9 RNA by RNA-immunoprecipitations (data not shown).

1. An isolated nucleic acid molecule comprising or consisting of a nucleotide sequence selected from the group consisting of:
i) the nucleotide sequence of SEQ ID NO: 1, SEQ ID NO: 3, SEQ ID NO: 5 or SEQ ID NO: 7;
ii) a nucleotide sequence wherein said sequence is degenerate as a result of the genetic code to the nucleotide sequence defined in (i);
iii) a nucleotide sequence that encodes the amino acid sequence of SEQ ID NO: 2, SEQ ID NO: 4, SEQ ID NO: 6 or SEQ ID NO: 8, wherein said amino acid sequence is modified by addition, deletion or substitution of at least one amino acid residue.

2. An isolated polypeptide comprising the amino acid sequence of SEQ ID NO: 2, SEQ ID NO: 4, SEQ ID NO: 6 or SEQ ID NO: 8, or a variant polypeptide wherein said variant polypeptide is modified by addition, deletion or substitution of at least one amino acid residue.

3. A vector that includes a nucleic acid molecule according to claim 1.

4. A cell transformed or transfected with a nucleic acid molecule according to claim 1.

5. The cell according to claim 4 wherein said cell is a eukaryotic cell.

6. The cell according to claim 5 wherein said eukaryotic cell is a mammalian cell.

7. The cell according to claim 4 wherein said cell is a prokaryotic cell.

8. The cell according to claim 7 wherein said prokaryotic cell is a microbial cell.

9. A small interfering RNA (siRNA) molecule wherein said siRNA molecule is specific for at least one SIRT 1 spliced variant comprising the nucleotide sequence of SEQ ID NO: 1, SEQ ID NO: 3, SEQ ID NO: 5 or SEQ ID NO: 7.

10. The siRNA according to claim 9 wherein said siRNA molecule specifically binds a splice junction of said spliced variant.

11. The siRNA according to claim 9 wherein said siRNA molecule comprises a first part that comprises a duplex ribonucleic acid (RNA) molecule and a second part that comprises a single stranded deoxyribonucleic acid (DNA) molecule wherein said single stranded DNA molecule comprises a 3′ terminal nucleic acid sequence wherein said sequence is adapted over at least part of its length to anneal by complementary base pairing to a part of said single stranded DNA to form a double stranded DNA structure.

12. The siRNA according to claim 9, wherein said siRNA molecule is selected from the group consisting of:
i)
ACUUUGCUGUAACCCUGUA;
ii)
UAAUUCCAAGUAAUCAGUA;
iii)
CACGGAUAGGAAAUAUAUC;
and
iv)
CCUUCUGUUCGUUCUUGUG

13. A pharmaceutical composition comprising a siRNA molecule according to claim 9 and including an excipient or carrier.

14. A method to diagnose cancer in a subject comprising:
i) providing an isolated biological sample to be tested;
ii) forming a preparation comprising said sample and one or more oligonucleotide primer pairs adapted to anneal to a nucleic acid molecule comprising the nucleic acid sequence of SEQ ID NO: 1, SEQ ID NO: 3, SEQ ID NO: 5 or SEQ ID NO: 7; a thermostable DNA polymerase, deoxynucleotide triphosphates and co-factors;
iii) providing polymerase chain reaction conditions sufficient to amplify said nucleic acid molecule(s);
iv) analysing the amplified products(s) of said polymerase chain reaction for the presence or absence of amplified product(s); and
v) comparing the amplified product(s) with a normal matched control.

15. The method according to claim 14 wherein the oligonucleotide primer pairs include oligonucleotide primers consisting of the nucleotide sequences:
i)
GGGATGGTATTTATGCTCGC
and
AAGAGGTGTGGGTGGCAACTCTG;
ii)
GGGATGGTATTTATGCTCGC
and
AACAGATACTGATTACTTGGA;
iii)
CCAAGGCCACGGATAGGAAAT
and
AAGAGGTGTGGGTGGCAACTCTG;
iv)
CCAAGGCCACGGATAGGAAAT
and
AACAGATACTGATTACTTGGA;
v)
ATAACCTTCTGTTCGTTCT
and
CTATGATTTGTTTGATGGATAGTTC;
vi)
CTAATTCCAAGTTCCATACCC
and
CTGAAGAATCTGGTGGTGAAG;
or
vii)
CCAAGGCCACGGATAGGAAAT
and
CTATGTTCTGGGTATAGTTGCG.

16. The method according to claim 14 wherein said comparison includes a quantitative and/or qualitative analysis of the expression of two or more SIRT 1 spliced variants relative to a normal matched control.

17. A kit comprising at least one variant specific primer pair selected from the group consisting of:
i)
GGGATGGTATTTATGCTCGC
and
AAGAGGTGTGGGTGGCAACTCTG;
ii)
GGGATGGTATTTATGCTCGC
and
AACAGATACTGATTACTTGGA;
iii)
CCAAGGCCACGGATAGGAAAT
and
AAGAGGTGTGGGTGGCAACTCTG;
iv)
CCAAGGCCACGGATAGGAAAT
and
AACAGATACTGATTACTTGGA;
v)
ATAACCTTCTGTTCGTTCT
and
CTATGATTTGTTTGATGGATAGTTC;
vi)
CTAATTCCAAGTTCCATACCC
and
CTGAAGAATCTGGTGGTGAAG;
and
vii)
CCAAGGCCACGGATAGGAAAT
and
CTATGTTCTGGGTATAGTTGCG.

18. The kit according to claim 17 wherein said kit further includes reagents required for polymerase chain reaction amplification of SIRT 1 spliced variant RNA.

19. A progenitor retinal pigmented epithelial (PRPE) cell which cell is modified wherein said modified cell has reduced or undetectable levels of SIRT 1.

20. The PRPE cell according to claim 19 wherein said cell is modified by transfection of a siRNA that reduces expression of SIRT 1.

21. The PRPE cell according to claim 19 wherein said siRNA is expressed by said PRPE cell.

22. The PRPE cell according to claim 19, wherein SIRT 1 is encoded by a nucleic acid molecule comprising the nucleotide sequence of SEQ ID NO: 9.

23. The PRPE cell according to claim 20 wherein said siRNA is designed with reference to the nucleotide sequence of SEQ ID NO: 9.

24. A method to enhance the differentiation of a progenitor retinal pigmented epithelial cell comprising:
i) providing a cell culture preparation comprising: a PRPE cell according to claim 19 and a cell culture medium; and
ii) providing cell culture conditions that enhance the differentiation of PRPE cells.

25. A method of differentiating PRPE cells comprising, contacting said cells with an agent that inhibits the expression or activity of SIRT 1.

26. The method according to claim 25 wherein said agent is a siRNA.
